(19) United States
(12) Patent Application Publication
(10) Pub. No.: US 2010/0181527 A1
(43) Pub. Date: Jul. 22, 2010

(54) NITROXIDE CONTAINING ELECTRODE MATERIALS FOR SECONDARY BATTERIES

(76) Inventors: Peter Nesvadba, Marly (CH); Lucienne Bugnon Folger, Pfeffingen (CH); Tobias Hintermann, Therwil (CH)

Correspondence Address: BASF Performance Products LLC;Patent Department, 540 White Plains Road, P.O. Box 2005, Tarrytown, NY, 10591, US

(21) Appl. No.: 12/664,420

(22) PCT Filed: Jun. 9, 2008
(86) PCT No.: PCT/EP08/57138

§ 371 Date: Dec. 14, 2009

(30) Foreign Application Priority Data
Jun. 19, 2007 (EP) 07110574.6

(51) Int. Cl. H01M 4/505; C07D 241/04; C07D 265/32

(52) U.S. Cl. 252/182.1; 544/385; 544/384; 544/173

(57) ABSTRACT

This invention relates to a stable secondary battery utilizing as active principle the oxidation and reduction cycle of a sterically hindered nitroxide radical, a sterically hindered oxoammonium cation, a sterically hindered hydroxylamine or a sterically hindered aminoxide anion containing a piperazin-2,6-dione, a piperazin-2-one or morpholin-2-one structural unit. Further aspects of the invention are a method for providing such a secondary battery, the use of the respective compounds as active elements in secondary batteries and selected novel compounds.

This invention relates to a stable secondary battery utilizing as active principle the oxidation and reduction cycle of a sterically hindered nitroxide radical, a sterically hindered oxoammonium cation, a sterically hindered hydroxylamine or a sterically hindered aminoxide anion containing a piperazin-2,6-dione, a piperazin-2-one or morpholin-2-one structural unit. Further aspects of the invention are a method for providing such a secondary battery, the use of the respective compounds as active elements in secondary batteries and selected novel compounds.

The use of various radicals, such as, for example, nitroxide radicals as active component in electrode materials of secondary batteries, in this case so called organic radical batteries, have been already disclosed in EP-A-1 128 453. Since low solubility or insolubility of the electrode material in the battery electrolyte is preferable, polymeric or oligomeric nitroxides are of particular interest.

Nitroxide polymers as cathode active materials in organic radical batteries have already been described. For example, Electrochimica Acta 50, 827 (2004) teaches the preparation of 4-methacryloyloxy-2,2,6,6-tetramethylpiperidine, its free radical polymerization and subsequent oxidation of the polymer into the corresponding polymeric nitroxide and its use in organic radical battery.

The underlying mechanism of energy storage in organic radical batteries is the reversible oxidation/reduction of the nitroxide radical according to Scheme 1:

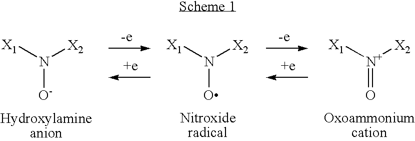

Even though the use of the full redox window (hydroxylamine anion <--> oxoammonium cation) is possible, the currently preferred organic radical batteries use the redox pair nitroxide radical <--> oxoammonium cation. Hence, the electrons are exchanged between the oxidized state N⁺═O and reduced state N—O..

Due to the fast growing market of electronic devices, such as mobile telephones and mobile personal computers (lap-tops), there have been increasing needs in the last years for small and large-capacity secondary batteries with high energy density.

Today the most frequently used secondary battery for such applications is the lithium-ion secondary battery. Such a lithium-ion secondary battery uses a transition-metal oxide containing lithium in the positive electrode (cathode) and carbon in a negative electrode (anode) as active materials, and performs charge and discharge via insertion of Li into and extraction of Li from these active materials.

There have been other attempts for developing a large-capacity secondary battery using an organic electrode material. For example, U.S. Pat. No. 2,715,778 has disclosed a secondary battery using an organic compound having a disulfide bond in a positive electrode, which utilizes, as a principle of a secondary battery, an electrochemical oxidation-reduction reaction associated with formation and dissociation of a disulfide bond.

The energy capacity of an organic radical battery achievable with a nitroxide material having a given concentration of redox active nitroxide groups can be increased if the redox potential E₀[V] of the nitroxide/oxoammonium couple is increased. Generally, if the redox potential of the nitroxide/oxoammonium couple is higher, the voltage of the corresponding organic radical battery is also higher.

However, the achievable specific energy capacity of nitroxide materials based on state of the art 2,2,6,6-tetramethylpiperidine-N-oxyl is still rather low.

Surprisingly it has now been found that a sterically hindered nitroxide radical, a sterically hindered oxoammonium cation, a sterically hindered hydroxylamine or a sterically hindered aminoxide anion containing a piperazin-2,6-dione, a piperazin-2-one or morpholin-2-one structural unit has generally a higher redox potential than state of the art compounds derived from 2,2,6,6-tetramethylpiperidine nitroxide.

Hence, they can be used for the preparation of electrodes, e.g. for secondary batteries or organic radical batteries, with a high specific energy capacity.

Additionally, if higher voltage is required by the device to be powered, it can be available directly with the compounds according to the invention without any expensive electronic circuitry or battery packs which would be required otherwise when using lower voltage cells.

Moreover, the compounds according to the invention generally show in a secondary battery fully reversible redox behaviour when subjected to repeated oxidation into the corresponding oxoammonium salts and back-reduction into the nitroxide. This reversibility is indeed a condition for applicability of a nitroxide as an active electrode material in a secondary battery. Moreover, the high redox reversibility of the nitroxide/oxoammonium couple assures a high cycling stability of the corresponding battery.

Sometimes, for example in Electrochimica Acta 2007, 52, 2153-2157, the terms “secondary battery”, “organic radical battery”, “supercapacitor” or “electrochemical supercapacitor” are used as synonyms. So the term secondary battery is to be understood to include organic radical battery and electrochemical supercapacitor and supercapacitor.

An aspect of the invention is a secondary battery, utilizing an electrode reaction of an active material in the reversible oxidation/reduction cycle in at least one of the positive or negative electrodes (for instance in the positive, for example in the negative electrode), which active material comprises a compound of formula Ia to Ic, preferably of formula Ia,

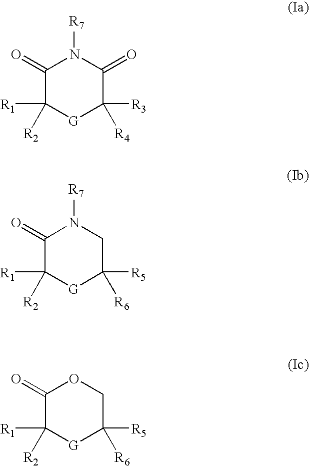

wherein G is >N—O., >N⁺═OAn⁻, >N—O⁻Li⁺ or >N—OH, preferably >N—O., >N⁺═OAn⁻, most preferably >N—O.;
R₁, R₂, R₃, R₄, R₅ and R₆ are independently CH₃ or C₂H₅, C₅-C₆-cycloalkyl, benzyl, phenyl or R₁ and R₂, R₃ and R₄ or R₅ and R₆ are independently together C₅- or C₆-cycloalkylidene,
or R₅ and R₆ form together with the linking carbon atom a

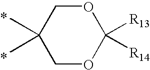

group, or R₅ is —CH₂—X—R₁₅;
R₇ is H, OH, —CN, -halogen, C₁-C₁₈alkyl, C₆-C₁₀aryl, C₇-C₁₁aralkyl, C₂-C₁₈alkenyl, C₂-C₁₈alkynyl, C₅-C₆cycloalkyl, glycidyl, —N₃, —NH₂, —NHR₈, —NR₈R₉, —CO—OR₈, —CO—R₈, —CO—NH—R₈, —CON(R₈)(R₉), —O—CO—R₈, —SR₈, —S(═O)R₈, —S(═O)₂R₈, —S—OR₈, —S(═O)OR₈, —S(═O)₂OR₈, —SiR₈R₉R₁₀, —S(═O)₂OR₁₁ or —PO(OR₁₁)(OR₁₂),
whereby or said alkyl, alkenyl, alkynyl and cycloalkyl are uninterrupted or interrupted by one or more heteroatomgroup, preferably by O, NR₈, Si(R₈)(R₉), PR₈ or S, most preferably by O or NR₈, or
whereby the said alkyl, alkenyl, alkynyl and cycloalkyl are unsubstituted or substituted by one or more heteroatomgroup, preferably by F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, —NCO, —N₃, NHC(O)NHR₈, —NR₈C(O)N(R₉)(R₁₀), —NHCOOR₁₀, —N(R₈)(R₉), —NR₈COOR₁₀, —N⁺(R₈)(R₉)(R₁₀) An⁻, S⁺(R₈)(R₉)An⁻ or P⁺(R₈)(R₉)(R₁₀)An⁻, or
whereby the said alkyl, alkenyl, alkynyl and cycloalkyl are interrupted by one or more heteroatomgroup (e.g. by O, NR₈, Si(R₈)(R₉), PR₈ or S, in particular by O or NR₈) and substituted by one or more heteroatomgroup (e.g. by F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, —NCO, —N₃, NHC(O)NHR₈, —NR₈C(O)N(R₉)(R₁₀), —NHCOOR₁₀, —N(R₈)(R₉), —NR₈COOR₁₀, —N⁺(R₈)(R₉)(R₁₀)An⁻, S⁺(R₈)(R₉)An⁻ or P⁺(R₈)(R₉)(R₁₀)An⁻), or
R₇ is a multivalent core with one or more structural units (Ia)-(Ic) attached, preferably the multivalent core is as defined below, or
R₇ is a 1,3,5-triazine core with 1, 2 or 3 structural units (Ia) attached;
R₈, R₉ and R₁₀ are independently H, C₁-C₁₈ alkyl, C₆-C₁₀aryl, C₇-C₁₁aralkyl, C₂-C₁₈alkenyl, C₂-C₁₈alkynyl, C₅-C₆cycloalkyl, C₄-C₁₂cycloalkenyl or C₅-C₁₂bicycloalkenyl;
R₁₁ and R₁₂ are independently H, NH₄, Li, Na, K or as defined for R₈;
R₁₃, R₁₄ are independently H or C₁-C₄ alkyl; or R₁₃ and R₁₄ form together with the linking carbon atom a C₄-C₈cycloalkylbiradical;
R₁₅ is H, C₁-C₁₈ alkyl, C₆-C₁₀aryl, C₇-C₁₁aralkyl, C₂-C₁₈alkenyl, C₂-C₁₈alkynyl, C₅-C₆cycloalkyl. glycidyl, —CO—OR₈, —CO—R₈, —CO—NH—R₈, —CON(R₈)(R₉), —S(═O)₂R₈, —S(═O)OR₈, —S(═O)₂OR₈, —SiR₈R₉R₁₀, —S(═O)₂OR₁₁ or —PO(OR₁₁)(OR₁₂),
whereby said alkyl, alkenyl, alkynyl and cycloalkyl are uninterrupted or interrupted by one or more heteroatomgroup, preferably by O, NR₈, Si(R₈)(R₉), PR₈ or S, most preferably by O or NR₈, or
whereby the said alkyl, alkenyl, alkynyl and cycloalkyl are unsubstituted or substituted by one or more heteroatomgroup, preferably by F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, —NCO, —N₃, NHC(O)NHR₈, —NR₈C(O)N(R₉)(R₁₀), —NHCOOR₁₀, —N(R₈)(R₉), —NR₈COOR₁₀, —N⁺(R₈)(R₉)(R₁₀) An⁻, S⁺(R₈)(R₉)An⁻ or P⁺(R₈)(R₉)(R₁₀)An⁻, or
whereby said alkyl, alkenyl, alkynyl and cycloalkyl are interrupted by one or more heteroatomgroup (e.g. by O, NR₈, Si(R₈)(R₉), PR₈ or S, especially by O or NR₈) and substituted by one or more heteroatomgroup (e.g. by F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, —NCO, —N₃, NHC(O)NHR₈, —NR₈C(O)N(R₉)(R₁₀), —NHCOOR₁₀, —N(R₈)(R₉), —NR₈COOR₁₀, —N⁺(R₈)(R₉)(R₁₀)An⁻, S⁺(R₈)(R₉)An⁻ or P⁺(R₈)(R₉)(R₁₀)An⁻),
or R₁₅ is a multivalent core with more than one structural units (Ib)-(Ic) attached, preferably the multivalent core is as defined below;
X is —O— or NR₁₆, preferably —O—;
R₁₆ is as defined for R₁₅;

An⁻ is an anion of an organic or inorganic acid, preferably anions derived from LiPF₆, LiClO₄, LiBF₄, LiO₃SCF₃, LiN(C₂F₅SO₂)₂, LiC(CF₃SO₂)₃, LiC(C₂F₅SO₂)₃, LiB(C₂O₄)₂, LiB(C₆H₅)₄, LiB(C₆F₅)₄, LiSbF₆, LiAsF₆, LiBr, LiBF₃C₂F₅ or LiPF₃(CF₂CF₃)₃, most preferably anions derived from LiPF₆, LiClO₄, LiBF₄, LiO₃SCF₃, LiN(C₂F₅SO₂)₂, LiC(CF₃SO₂)₃, LiC(C₂F₅SO₂)₃ or LiB(C₂O₄)₂;

with the proviso that
R₇ does not contain a 1,3,5-triazine core for compounds of formula Ib.

For instance, R₇, R₁₅ and R₁₆ do not contain conductive carbon selected from the group consisting of single walled carbon nanotubes, multiwalled carbon nanotubes, carbon nanofibers, carbon fibers, fullerenes, graphite, graphene, carbon black and glassy carbon; and

R₇, R₁₅ and R₁₆ do not contain an aromatic carbocyclic ring system of at least two aromatic rings.

Examples of an aromatic carbocyclic ring system of at least two aromatic rings are perylenyl, naphthyl, anthracenyl, phenanthrenyl, fluoranthenyl, pyrenyl, chrysenyl, benzanthracenyl, biphenyl, terphenyl, dibenzanthracenyl, benzofluoranthenyl, benzopyrenyl, indenopyrenyl and benzoperylenyl.

For instance, the compound of formula Ia to Ic does not comprise a 1,3,5-triazine core. The 1,3,5-triazine core corresponds to the formula

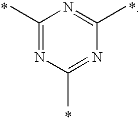

For instance, only one of R₇, R₁₅ and R₁₆ is a multivalent core or 1,3,5-triazine core.

For example, the alkyl, alkenyl, alkynyl and cycloalkyl mentioned herein are unsubstituted and uninterrupted.

An electrode active material as used herein refers to a material directly contributing to an electrode reaction such as charge and discharge reactions, and plays a main role in a secondary battery system. An active material in this invention may be used as either a positive electrode or negative electrode active material, but it may be more preferably used as a positive electrode active material because it is characterized by a light weight and has a good energy density and safety in comparison with a metal oxide system.

The counter ion of the oxoammonium cation, An⁻ may be, for example, the anion derived from LiPF₆, LiClO₄, LiBF₄, LiO₃SCF₃, LiN(C₂F₅SO₂)₂, LiC(CF₃SO₂)₃, LiC(C₂F₅SO₂)₃, LiB(C₂O₄)₂, LiB(C₆H₅)₄, LiB(C₆F₅)₄, LiSbF₆, LiAsF₆, LiBr, LiBF₃C₂F₅ or LiPF₃(CF₂CF₃)₃.

For instance, the multivalent core of R₇, R₁₅ and R₁₆ is a C₂-C₂₀ polyacyl of a di-, tri-, tetra-, penta- or hexa-carboxylic acid (e.g. aliphatic carboxylic acid, in particular a saturated, acyclic carboxylic acid), C₂-C₂₀ alkyl, C₆-C₁₀aryl (e.g. phenyl), C₅-C₉heteroaryl, whereby the said polyacyl and alkyl are uninterrupted or interrupted by one or more heteroatomgroup, preferably by O, NR₈, Si(R₈)(R₉), PR₈ or S, most preferably by O or NR₈, or whereby the said polyacyl and alkyl are unsubstituted or substituted by one or more heteroatomgroup, preferably by F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, —NCO, —N₃, NHC(O)NHR₈, —NR₈C(O)N(R₉)(R₁₀), —NHCOOR₈, —N(R₈)(R₉), —NR₈COOR₉, —N⁺(R₈)(R₉)(R₁₀) An⁻, S⁺(R₈)(R₉)An⁻ or P⁺(R₈)(R₉)(R₁₀)An⁻, or

whereby the said polyacyl and alkyl are interrupted by one or more heteroatomgroup (e.g. by O, NR₈, Si (R₈)(R₉), PR₈ or S, especially by O or NR₈) and substituted by one or more heteroatomgroup (e.g. by F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, —NCO, —N₃, NHC(O)NHR₈, —NR₈C(O)N(R₉)(R₁₀), —NHCOOR₈, —N(R₈)(R₉), —NR₈COOR₉, —N⁺(R₈)(R₉)(R₁₀)An⁻, S⁺(R₈)(R₉)An⁻ or P⁺(R₈)(R₉)(R₁₀)An⁻); or
the compounds of formula Ia to Ic correspond to compounds of formula Ia1 to Ic7

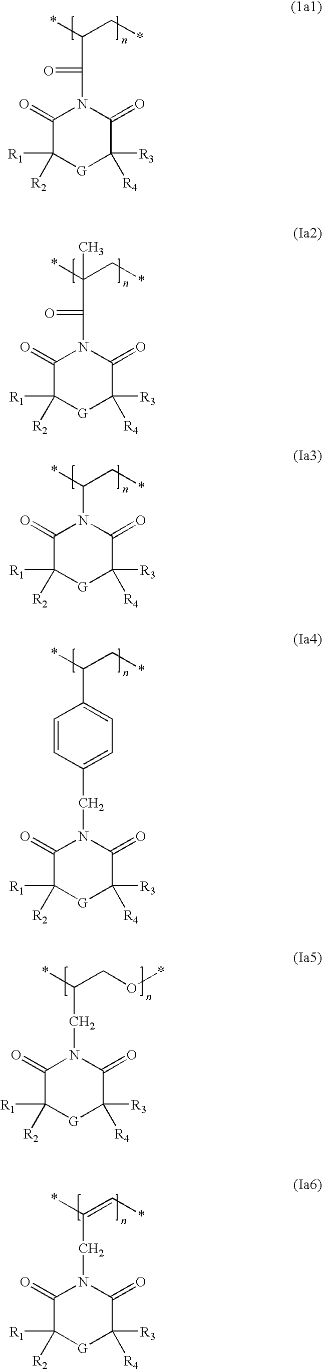

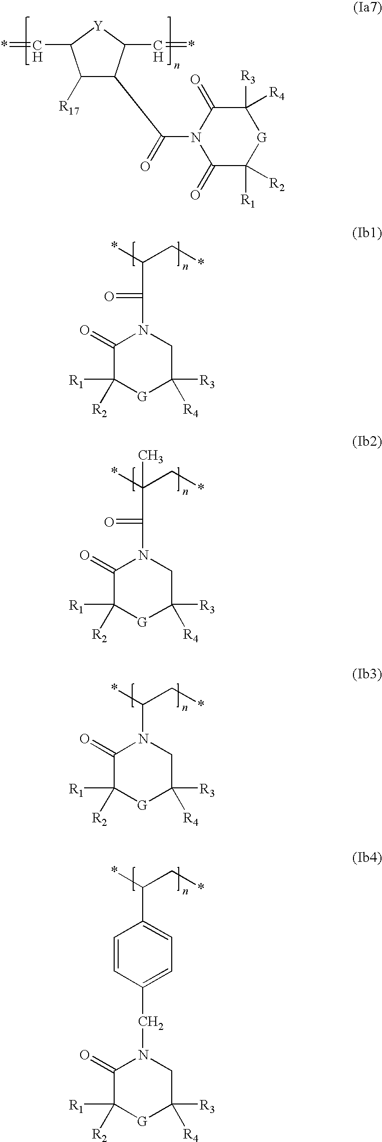

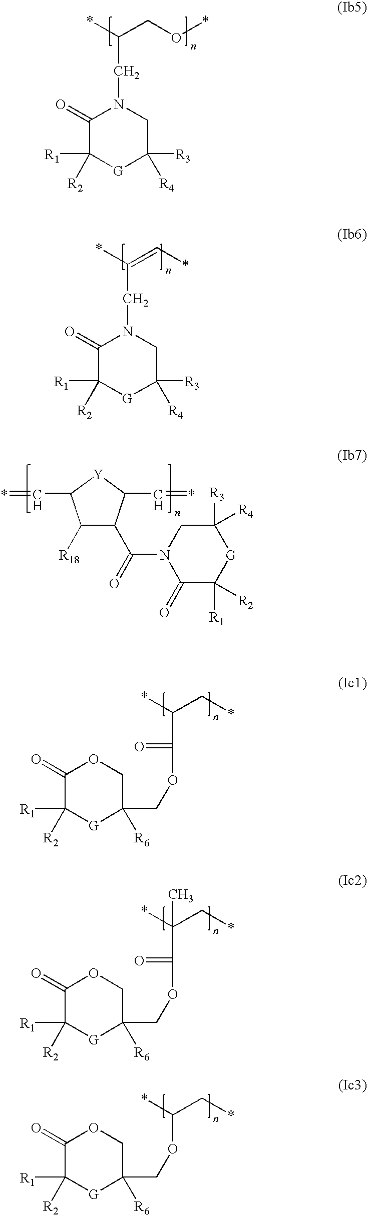

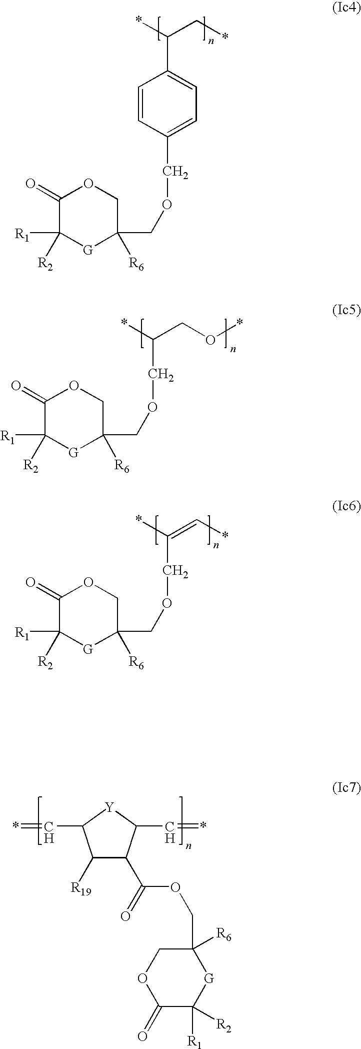

R₁₇ is H or a group

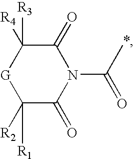

preferably

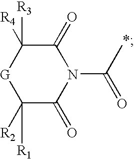

R₁₈ is H or a group

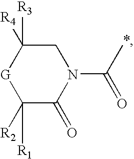

preferably

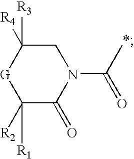

R₁₉ is H or a group

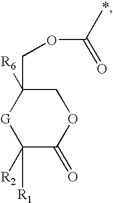

preferably

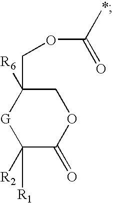

Y is —CH₂— or —O—, preferably —CH₂—; and
n is 2-100 000, preferably 10-10 000, more preferably 20-1000, most preferably 20-200.

For example, the alkyl and polyacyl mentioned herein are unsubstituted and uninterrupted.

For example, copolymers comprising monomers bearing a group Ia′ to Ic′

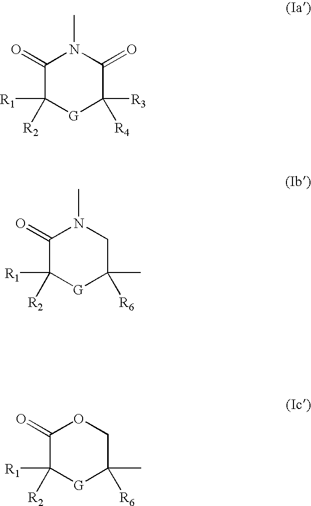

of above mentioned polymers are the object of the present invention. Moreover, these monomers can be copolymerized with other polymerizable monomers such as derivatives of acrylic or methacrylic acid, acrylonitrile, styrene, oxiranes, oxetanes, vinyl derivatives, acetylenes or monomers amenable to Ring Opening Metathesis Polymerization (ROMP). It is also understood that these monomers can bear nitroxide radicals known in the prior art, in particular 2,2,6,6-tetramethylpiperidin-N-oxyl.

For instance, compounds of formula Ia1 to Ic7 is a copolymer with a monomer bearing 2,2,6,6-tetramethylpiperidin-N-oxyl, in particular a free valence of a compound of formula Ia1 to Ic7 is occupied with a polymer bearing 2,2,6,6-tetramethylpiperidin-N-oxyl having m repeating units such as

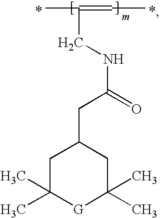

especially such a copolymer is

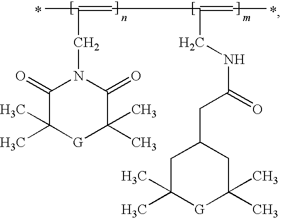

wherein m is as defined for n.

For instance, R₁, R₂, R₃, R₄, R₅ and R₆ are independently CH₃ or C₂H₅, or

R₁ and R₂, R₃ and R₄ or R₅ and R₆ are independently together C₆-cycloalkylidene, or

### R₅ is CH₂—X—R₁₅;

R₇ is H, OH, —CN, C₁-C₆ alkyl, phenyl, benzyl, C₂-C₆alkenyl, C₂-C₆alkynyl, C₅-C₆cycloalkyl, glycidyl, —CO—OR₈, —CO—R₈, —CO—NH—R₈, —CON(R₈)(R₉), —S(═O)₂R₈, —S(═O)₂OR₈ or —PO(OR₁₁)(OR₁₂),
whereby the said alkyl, alkenyl, alkynyl and cycloalkyl are uninterrupted or interrupted by one or more O, NR₈, or S, preferably by O or NR₈, or
whereby said alkyl, alkenyl, alkynyl or cycloalkyl are unsubstituted or substituted by one or more F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, NHC(O)NHR₈, —NR₈C(O)N(R₉)(R₁₀), —NHCOOR₁₀, —N(R₈)(R₉) or —NR₈COOR₁₀; or
whereby the said alkyl, alkenyl, alkynyl and cycloalkyl are interrupted by one or more O, NR₈, or S (e.g. by O or NR₈) and substituted by one or more F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, NHC(O)NHR₈, —NR₈C(O)N(R₉)(R₁₀), —NHCOOR₁₀, —N(R₈)(R₉) or —NR₈COOR₁₀;
or R₇ is a multivalent core with more than one structural units (Ib)-(Ic) attached, whereby the multivalent core is a C₂-C₈ polyacyl from di-, tri-, tetra-, penta- or hexa-carboxylic acid (e.g. aliphatic carboxylic acid, in particular a saturated, acyclic carboxylic acid), C₂-C₈ alkyl or phenyl,
R₈, R₉ and R₁₀ are independently H, C₁-C₆ alkyl, phenyl, benzyl, C₂-C₄alkenyl, C₂-C₄alkynyl, C₅-C₆cycloalkyl, C₄-C₆cycloalkenyl or C₅-C₇bicycloalkenyl;
R₁₁ and R₁₂ are independently H, Li, Na, K or as defined for R₈; and
R₁₅ is H, OH, —CN, C₁-C₆ alkyl, phenyl, benzyl, C₂-C₆alkenyl, C₂-C₆alkynyl, C₅-C₆cycloalkyl, glycidyl, —CO—OR₈, —CO—R₈, —CO—NH—R₈, —CON(R₈)(R₉), —S(═O)₂R₈, —S(═O)₂OR₈ or —PO(OR₁₁)(OR₁₂),
whereby the said alkyl, alkenyl, alkynyl and cycloalkyl are uninterrupted or interrupted by one or more O, NR₈, or S, preferably by O or NR₈, or
whereby said alkyl, alkenyl, alkynyl or cycloalkyl are unsubstituted or substituted by one or more F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, NHC(O)NHR₈, —NR₈C(O)N(R₉)(R₁₀), —NHCOOR₁₀, —N(R₈)(R₉) or —NR₈COOR₁₀; or
whereby the said alkyl, alkenyl, alkynyl and cycloalkyl are interrupted by one or more O, NR₈, or S (e.g. by O or NR₈) and substituted by one or more F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, NHC(O)NHR₈, —NR₈C(O)N(R₉)(R₁₀), —NHCOOR₁₀, —N(R₈)(R₉) or —NR₈COOR₁₀; or
the compounds of formula Ia to Ic correspond to compounds of formula Ia1 to Ic7.

For example, R₇ is H, C₁-C₆ alkyl, phenyl, benzyl, C₂-C₃alkenyl, C₂-C₃alkynyl, glycidyl, —CO—OR₈, —CO—R₈, —CO—NH—R₈, —CON(R₈)(R₉), —S(═O)₂R₈, —S(═O)₂OR₁₁ or —PO(OR₁₁)(OR₁₂), whereby said alkyl, alkenyl and alkynyl are unsubstituted or substituted by one or more —COOR₈, —CONHR₈, —CON(R₈)(R₉), OR₈, —OC(O)R₈, —OC(O)OR₈, —NHC(O)R₈, —NR₈C(O)R₉ or —N(R₈)(R₉),

or R₇ is a multivalent core with more than one structural units (Ia) attached, whereby the multivalent core is a C₂-C₈ polyacyl from di-, tri-, tetra-, penta- or hexa-carboxylic acid (e.g. aliphatic carboxylic acid, in particular a saturated, acyclic carboxylic acid) or C₂-C₈ alkyl;
R₈, R₉ and R₁₀ are independently H, C₁-C₆ alkyl, phenyl, benzyl, C₂-C₄alkenyl, C₂-C₄alkynyl, C₅-C₆cycloalkyl, C₄-C₆cycloalkenyl or C₅-C₇bicycloalkenyl; and
R₁₁ and R₁₂ are independently H, Li, Na, K or as defined for R₈; and
R₁₅ is H, C₁-C₆ alkyl, phenyl, benzyl, C₂-C₃alkenyl, C₂-C₃alkynyl, glycidyl, —CO—OR₈, —CO—R₈, —CO—NH—R₈, —CON(R₈)(R₉), —S(═O)₂R₈, —S(═O)₂OR₁₁ or —PO(OR₁₁)(OR₁₂),
whereby said alkyl, alkenyl and alkynyl are unsubstituted or substituted by one or more —COOR₈, —CONHR₈, —CON(R₈)(R₉), OR₈, —OC(O)R₈, —OC(O)OR₈, —NHC(O)R₈, —NR₈C(O)R₉ or —N(R₈)(R₉),
preferably R₁₅ is H, C₁-C₆ alkyl, phenyl, benzyl, C₂-C₃alkenyl, C₂-C₃alkynyl, glycidyl, —CO—OR₈ or —CO—R₈.

For instance, R₁, R₂, R₃, R₄, R₅ and R₆ are independently CH₃ or C₂H₅, or

R₅ is —CH₂—O—CO—C₁-C₆alkyl; preferably R₁, R₂, R₃, R₄, R₅ and R₆ are CH₃;
R₇ is H, C₁-C₆ alkyl, phenyl, benzyl, C₂-C₃alkenyl, C₂-C₃alkynyl, —CO—OR₈ or —CO—R₈,
whereby said alkyl, alkenyl, alkynyl are unsubstituted or substituted (e.g. unsubstituted) by one or more —COOR₈, OR₈ or —OC(O)R₈;
R₈ is H, C₁-C₆ alkyl, phenyl, benzyl, C₂-C₄alkenyl or C₂-C₄alkynyl.

For example, the compound of formula Ia to Ic correspond to a compound of formula Ia1, Ia2, Ia5, Ia6, Ia7, Ib1, Ib2, Ib5, Ib6 or Ib7, in particular Ia1, Ia2, Ia5, Ia6, Ia7 or Ib6, especially Ia6 or Ib6.

For instance, R₁, R₂, R₃, R₄, R₅ and R₆ are independently CH₃ or C₂H₅, or

R₅ is —CH₂—O—OC—C₁-C₆alkyl; preferably R₁, R₂, R₃, R₄, R₅ and R₆ are CH₃;
R₇ is H, C₁-C₆ alkyl, phenyl, C₂-C₃alkynyl or —CO—C₁-C₆alkyl;
or
the compounds of formula Ia or Ib correspond to compounds of formula Ia1, Ia2, Ia5, Ia6, Ia7 or Ib6 (e.g. Ia6 or Ib6), wherein
R₁-R₄ are as defined above,

### R₁₇ is

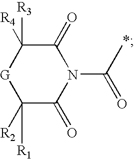

and

### Y is —CH₂—.

When a denotation (e.g. R₈, R₉ or R₁₀) occurs more than once (e.g. twice) in a compound, this denotation may be different groups or the same group.

It is to be understood that alkyl interrupted by a heteroatomgroup comprises at least 2 carbon atoms, that alkenyl and alkynyl interrupted by a heteroatomgroup comprises at least 3 carbon atoms and that polyacyl substituted by a heteroatomgroup comprises at least 3 carbon atoms.

In the definitions the term alkyl comprises within the given limits of carbon atoms, for example methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, 2-methylheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl or dodecyl.

Examples of alkenyl are within the given limits of carbon atoms vinyl, allyl, 1-methylethenyl, and the branched and unbranched isomers of butenyl, pentenyl, hexenyl, heptenyl, octenyl, nonenyl, decenyl, undecenyl and dodecenyl. The term alkenyl also comprises residues with more than one double bond that may be conjugated or non-conjugated, for example may comprise one double bond.

Examples of alkynyl are within the given limits of carbon atoms ethynyl, propargyl, 1-methylethynyl, and the branched and unbranched isomers of butynyl, pentynyl, hexynyl, heptynyl, octynyl, nonynyl, decynyl, undecynyl and dodecynyl, especially propargyl. The term alkynyl also comprises residues with more than one triple bond and residues with a triple bond and a double bond, all of which may be conjugated or non-conjugated. For instance, alkynyl comprises one triple bond.

Some examples of cycloalkyl are cyclopentyl, cyclohexyl, methylcyclopentyl, for instance cyclohexyl.

Examples of cycloalkylidene are cyclopentylidene and cyclohexylidene.

Examples of cycloalkylbiradicals are cyclopentylbiradicals and cyclohexylbiradicals.

Some examples of cycloalkenyl are cyclopentenyl, cyclohexenyl, methylcyclopentenyl, dimethylcyclopentenyl and methylcyclohexenyl. Cycloalkenyl may comprise more than one double bond that may be conjugated or non-conjugated, for example may comprise one double bond.

Aryl is for example phenyl.

Phenylalkyl is for instance benzyl or α,α-dimethylbenzyl.

The term halogen may comprise chlorine, bromine and iodine; for example halogen is chlorine.

Examples of bicycloalkenyl are norbornenyl and norborna-2,5-dienyl.

Examples of a polyacyl are oxalic acid, malonic acid, succinic acid, glutaric acid, adipic acid, pimelic acid, azelaic acid, decanedioic acid, maleic acid, fumaric acid, itaconic acid, phathalic acid, terephthalic acid, 1,2,3-propanetricarboxylic acid, hemimellitic acid, trimellitic acid and trimesic acid.

Examples of heteroaryl are piperidinyl, 9H-purinyl, pteridinyl, chinolinyl, isochinyl, acridinyl and phenazinyl.

The preparation of piperazin-2,6-diones (Ia) and of related nitroxide radicals is described for example in: Ramey, Ch. E.; Luzzi, J. J.: Ger. Offen. (1973), DE 2314091.

The preparation of piperazin-2-ones (Ib) and of related nitroxide radicals is described for example in: Lai, J. T. Synthesis (1981), 40.

The preparation of morpholin-2-ones (Ic) and of related nitroxide radicals is described for example in: Lai, J. T.; Masler, W. F.; Nicholas, P. P.; Pourahmady, N.; Puts, R. D.; Tahiliani, S.: Eur. Pat. Appl. (1998), EP 869137 A1

Further examples of piperazin-2-ones, morpholin-2-ones and of related nitroxide radicals are described for example in Nesvadba, P.; Kramer, A.; Zink, M.-O.; Ger. Offen. (2000), DE 19949352 A1.

Further functionalization of the piperazin-2,6-diones (Ia) piperazin-2-ones (Ib) or morpholin-2-ones (Ic) building blocks into the compounds according to the present invention uses standard methods of organic synthesis which are described in countless textbooks, for example in “Organikum, Wiley-VCH, 2001.”

The introduction of the radical function into the pertinent molecules can be performed at diverse stages of the synthesis.

It is for example possible that simple piperazin-2,6-diones, piperazin-2-ones or morpholin-2-ones nitroxide building blocks are first made and then further evaluated into more complex structures. An example of this approach is the synthesis of Cmpd 5 (Table 1, Example 5) from the nitroxide Cmpd 4:

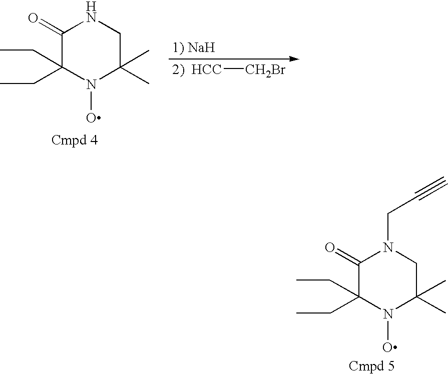

However, the nitroxide function can be introduced also in a very last step of the synthesis as exemplified on the preparation of Cmpd 3 (Table 1, Cmpd 3):

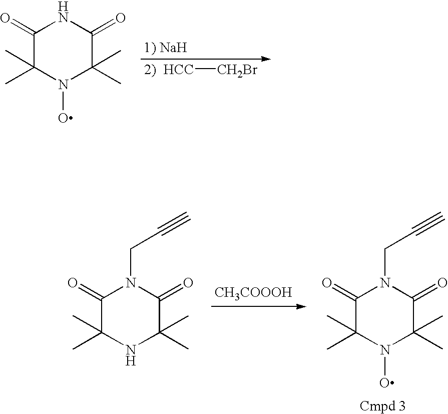

The preparation of the pertinent polymeric structures can be performed using known methods, as described for example in “Principles of Polymerization, G. Odian, J. Wiley & Sons, 1991.”

The methodology for preparation of polymers via Ring Opening Polymerization (ROMP) is described for example in “Olefin Metathesis and Metathesis Polymerization, K. J. Ivin, J. C. Mol, Academic Press, 1997.”

The polyacetylene polymers can be made via methods described by M. G. Mayershofer, O. Nuyken.: J. Polym. Sci. A, Polym. Chem. 43, 5723-5747 (2005).

The nitroxide monomers amenable to ROMP or acetylene nitroxide monomers can be also photopolymerized using the methodology and catalyst described by: Van der Schaaf, P.; Hafner, A.; Muhlebach, A. WO 96/19540, (1996).

The nitroxide function can be introduced into the polymers via their oxidation as a last step. Suitable oxidation agents are for example hydrogen peroxide, peracetic or performic acids, or t-butyl hydroperoxide.

Nitroxide polymers can be also prepared directly from the corresponding nitroxide containing monomers if the polymerization mechanism tolerates the presence of a nitroxide functionality. This is for example the case for ROMP or acetylene polymerization which is exemplified on the preparations of Cmpds 10 and 11 (Table 1, Examples 10, 11):

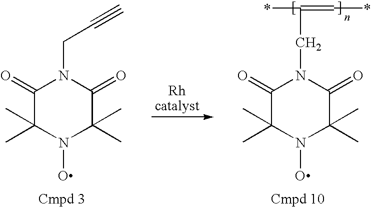

A suitable polymerization method for acrylic or methacrylic monomers bearing nitroxide groups is anionic or group transfer polymerization (GTP). The application of GTP for polymerization of nitroxide bearing monomers was described by: Nesvadba, P. Bugnon, L. WO 2006/131451 A1.

Vinyl ether monomers bearing nitroxides can be polymerized via cationic polymerization.

The compounds of formula Ia to Ic with G being >N⁺═OAn⁻ can be prepared via oxidation of the corresponding nitroxide radicals as described for example by: J. M. Bobbitt, M. C. L. Flores.: Heterocycles, 27 (2), pp 509-533 (1988).

The compounds of formula Ia to Ic with G being >N—O⁻Li⁺ can be prepared via deprotonation of the corresponding hydroxylamines with strong Lithium bases, for example lithiumdiisopropylamide (LDA), LiH or metallic Lithium or via one-electron reduction of nitroxide radicals with metallic lithium. They can also be prepared in analogous reactions with metallic sodium or potassium are described, for example by: B. Moon, M. Kang.: Macromol. Res. 13(3), pp 229-235 (2005).

The compound of formula Ia to Ic with G being >N—OH (hydroxylamines) can be made via reduction of the corresponding nitroxides with broad variety of reducing agents such as for example sodium ascorbate (H. Henry-Riyad, T. Tidwell.: Journal of Physical Organic Chemistry 16(8), pp 559-563 (2003)) or hydrogen gas with Platinum catalyst.

Another possibility for the preparation of the hydroxylamines is the partial oxidation of the corresponding amines >NH with, for example dimethyldioxirane, as described by: R. W. Murray, M. Singh.: Synthetic Communications 19(20), pp 3509-22 (1989).

The polymers according to the present invention may be optionally crosslinked.

The suitable crosslinking method depends on the type of monomer and on the polymerization chemistry.

One possibility is to use a suitable polyfunctional monomer as a crosslinking additive which is added into the polymerizing mixture. This approach is exemplified on the preparation of the crosslinked Cmpd 10 which uses N,N′-dipropargyloxalamide as crosslinking agent:

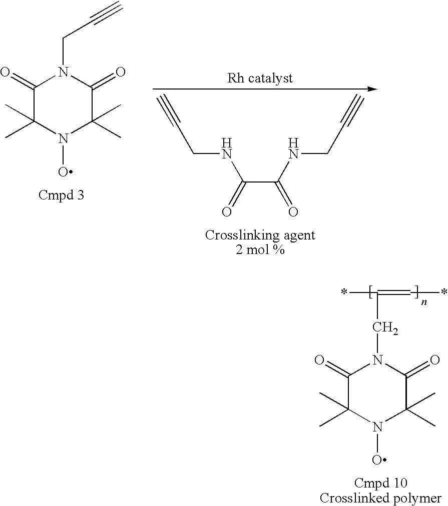

The amount of the crosslinking agent can vary broadly, depending on the desirable crosslinking density. Variation of the crosslinking density allows fine tuning of the swelling behaviour of the polymer and of its mechanical properties.

Clearly, the kind of the crosslinking agent will depend on the involved polymerization chemistry.

Thus, polyfunctional acrylester or acrylamides or methacrylester or methacrylamides may be used for crosslinking of acrylic or methacrylic nitroxide bearing monomers. Non limiting examples are:
Bispenol A dimethacrylate,
Trimethylolpropane trimethacrylate,
Ethylene glycol dimethacrylate,
Triethylene glycol dimethacrylate,
1,3-Propane diol dimethacrylate,
1,2-Propane diol dimethacrylate,
1,4-Butandiol dimethacrylate,
1,3-Butandiol dimethacrylate,
Diethylene glycol dimethacrylate,
Tetraethylene glycol dimethacrylate,
1,6-Hexandiol dimethacrylate,
Neopentylglykol dimethacrylate,
1,4-Cyclohexane diol dimethacrylate,
Glyceryl trimethacrylate,
1,1,1-Trimethylol ethane trimethacrylate,
Tris-hydroxyethyl-isocyanurate trimethacrylate,
Pentaerythritol tetramethacrylate,

Polyfunctional vinyl ether can be used for crosslinking of vinyl ether nitroxide bearing monomers. Non limiting examples are:
1,4-Butandiol divinyl ether,
Diethylene glycol divinyl ether,
Triethylene glycol divinyl ether,
or 1,4-Cyclohexane dimethanol divinyl ether.

Polyfunctional ester of propargyl alcohol or polyfunctional amides derived from propargyl amine can be used for crosslinking of acetylene type nitroxide monomers. Non limiting examples are:
Oxalic acid di-propargyl ester
Oxalic acid di-propargyl amid
Adipic acid di-propargyl ester
Adipic acid di-propargyl amid
1,3,5-Benzene-tricarboxylic acid tri-propargyl ester
1,3,5-Benzene-tricarboxylic acid tri-propargyl amide

Polyfunctional derivatives of norbornene can be used for crosslinking of ROMP-amenable nitroxide monomers. Non limiting examples are:
Diester of ethylene glycol with 5-norbornene-2-carboxylic acid
Diester of hexane diol with 5-norbornene-2-carboxylic acid
Diamide of ethylene diamine with 5-norbornene-2-carboxylic acid
Diamide of hexamethylene diamine with 5-norbornene-2-carboxylic acid
Tetraester of pentaerythritol with 5-norbornene-2-carboxylic acid

It is also possible to crosslink the soluble nitroxide bearing polymer. One attractive possibility is the photocrosslinking of nitroxide polymers having unsaturated bonds in the polymeric backbone. Examples of such polymers are the polyacetylene polymers (Ia6), (Ib6) and (Ic6) or the ROMP polymers (Ia7), (Ib7), (Ic7). The suitable photocrosslinking agents are bis-azide compounds such as for example:
2,6-Bis(4-azidobenzylidene)-cyclohexanone
2,6-Bis(4-azidobenzylidene)-4-methylcyclohexanone
2,6-Bis(4-azidobenzylidene)-4-t-butylcyclohexanone
p-Azidophenyl sulfone
4,4′-Diazidodiphenyl ether

This photocrosslinking method is particularly suitable for production of thin layer or microstructured nitroxide polymer cathodes. The manufacturing process can use spin coating, ink-jet printing or roll-to-roll printing of the soluble nitroxide polymer containing the suitable photocrosslinking reagent followed by the photocrosslinking.

Such thin polymer nitroxide cathodes are of interest in diverse application such as active RFID tags, printable or wearable electronics.

Yet another possibilty of crosslinking uses a reaction of the soluble nitroxide polymer with bis-functional or polyfunctional organic halogen containing compound in the presence of a transition metal or transition metal salt in its lower oxidation state and optionally a ligand, capable of complexing the transition metal or transition metal salt, for instance as outlined in WO2007/115939.

Preferred is a secondary battery, wherein the electrode reaction is that in the positive electrode.

Preferred is a secondary battery, wherein G is

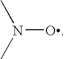

For instance, G is

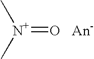

when the respective electrode is in the charged state.

In this invention, a binder may be used for reinforcing binding between components.

Examples of a binder include polyvinylidene fluoride, a copolymer of vinylidene fluoride and hexafluoropropylene, a copolymer of vinylidene fluoride and tetrafluoroethylene, polytetrafluoroethylene, a copolymer rubber of styrene and butadiene, and resin binders such as polypropylene, polyethylene and polyimide.

The active material in at least one of a positive electrode and a negative electrode comprises, without restrictions to its amount, a compound of formula Ia to Ic. However, since the capacity as a secondary battery depends on the amount of the compound of formula Ia to Ic contained in the electrode, the content is desirably 10 to 100% by weight, preferably 20 to 100% and in particular 50 to 100% for achieving adequate effects.

It is also possible to use more than one compound of formula Ia to Ic as active electrode material. The compound of formula Ia to Ic may be mixed, for example, with a known active material to function as a complex active material.

When using the instant compound of formula Ia to Ic in a positive electrode, examples of materials for the negative electrode layer include carbon materials such as graphite and amorphous carbon, lithium metal or a lithium alloy, lithium-ion occluding carbon and conductive polymers. These materials may take an appropriate form such as film, bulk, granulated powder, fiber and flake.

A conductive auxiliary material or ion-conductive auxiliary material may also be added for reducing impedance during forming the electrode layer. Examples of such a material include carbonaceous particles such as graphite, carbon fibers, carbon black and acetylene black and conductive polymers such as polyaniline, polypyrrole, polythiophene, polyacetylene and polyacene as conductive auxiliary materials as well as a gel electrolyte and a solid electrolyte as ion-conductive auxiliary material.

For instance, the active material comprises a blend of at least one compound of formula Ia to Ic and a further active material selected from the group consisting of an organic radical (e.g. a nitroxyl radical) different from those described herein, LiFePO₄, Li₂FeSiO₄, Li_{w}MnO₂, MnO₂, Li₄Ti₅O₁₂, LiMnPO₄, LiCoO₂, LiNiO₂, LiNi₁₋ₓCo_{y}Met_{z}O₂, LiMn_{0.5}Ni_{0.5}O₂, LiMn_{0.3}Co_{0.3}Ni_{0.3}O₂, LiFeO₂, LiMet_{0.5}Mn_{1.5}O₄, vanadium oxide, Li₁₊ₓMn_{2-z}Met_{y}O₄₋ₘXₙ, FeS₂, LiCoPO₄, Li₂FeS₂, Li₂FeSiO₄, LiMn₂O₄, LiNiPO₄, LiV₃O₄, LiV₆O₁₃, LiVOPO₄, LiVOPO₄F, Li₃V₂(PO₄)₃, MoS₃, sulfur, TiS₂, TiS₃ and combinations thereof, whereby 0<m<0.5, 0<n<0.5, 0.3≦w≦0.4, 0<x<0.3, 0<z<0.5, 0<y<0.5, Met is Al, Mg, Ti, B, Ga, Si, Ni, or Co, and X is S or F; preferably a blend of at least one compound of formula Ia to Ic and a further active material selected from the group consisting an organic radical (e.g. a nitroxyl radical) different from those described herein, LiFePO₄, MnO₂, Li₄Ti₅O₁₂, LiMnPO₄, LiCoO₂, LiMn_{0.5}Ni_{0.5}O₂, LiMn_{0.3}Co_{0.3}Ni_{O0.3}O₂, vanadium oxide, FeS₂, LiMn₂O₄, LiV₃O₄, LiV₆O₁₃, sulfur, TiS₂, TiS₃ and combinations thereof,

more preferably a blend of at least one compound of formula Ia to Ic and a further active material selected from the group consisting an organic radical (e.g. a nitroxyl radical) different from those described herein, LiFePO₄, LiMnPO₄, LiCoO₂, LiMn_{0.3}Co_{0.3}Ni_{0.3}O₂, LiMn₂O₄, and combinations thereof, for instance LiFePO₄, LiMnPO₄, LiCoO₂, LiMn_{0.3}Co_{0.3}Ni_{0.3}O₂, LiMn₂O₄, and combinations thereof,
for example a blend of at least one compound of formula Ia to Ic and a further active material selected from the group consisting an organic radical (e.g. a nitroxyl radical) different from those described herein.

Examples of organic radicals different from those described herein are as outlined in EP-A-1128453. More particularly, the organic radical can be as represented in EP-A-1128453 as chemical formula (A1)-(A11), especially as chemical formula (A2) and (A7)-(A10), in particular as chemical formula (A7)-(A10). Further examples of organic radicals different from those described herein are as outlined in WO-A-2006/131451. More particularly, the organic radical can be a nitroxide containing polymer as outlined on page 3, line 21 to page 4, line 2. Further examples of organic radicals different from those described herein are as outlined in T. Katsumata, et al., Macromolecular Rapid Communications 2006, 27, 1206-1211. More particularly, the organic radical can be a nitroxide containing polymer denoted as poly(1)-poly(3) and as poly(4) and poly(5) as outlined on page 1207.

For instance, the weight ratio of a compound of formula Ia to Ic as defined herein to a further active material is 1:9 to 100:1, preferably 1:9 to 10:1, more preferably 1:5 to 5:1, most preferably 1:5 to 2:1.

A catalyst may also be used for accelerating the electrode reaction. Examples of a catalyst include conductive polymers such as polyaniline, polypyrrole, polythiophene, polyacetylene and polyacene; basic compounds such as pyridine derivatives, pyrrolidone derivatives, benzimidazole derivatives, benzothiazole derivatives and acridine derivatives; and metal-ion complexes.

In general, a radical concentration may be expressed as a spin concentration. That is, a spin concentration means the number of unpaired electrons (radicals) per unit weight, which is determined by, for example, the following procedure from an absorption area intensity in an electron spin resonance spectrum (hereinafter, referred to as an “ESR” spectrum). First, a sample to be measured by ESR spectroscopy is pulverized by grinding it in, for example, a mortar, whereby the sample may be ground to a particle size in which skin effect, i.e., a phenomenon that microwave does not penetrate a sample, can be ignored. A given amount of the pulverized sample is filled in a quartz glass capillary with an inner diameter of 2 mm or less, preferably 1 to 0.5 mm, vacuumed to 10−5 mm Hg or less, sealed and subjected to ESR spectroscopy. ESR spectroscopy may be conducted in any commercially available model. A spin concentration may be determined by integrating twice an ESR signal obtained and comparing it to a calibration curve. There are no restrictions to a spectrometer or measuring conditions as long as a spin concentration can be accurately determined. For the stability of a secondary battery, a radical compound is desirably stable. A stable radical as used herein refers to a compound whose radical form has a long life time.

The concentration of the radical compound, i.e. a compound of formula Ia to Ic with G being >N—O., in this invention is preferably kept to 10¹⁹ spin/g or more, more preferably 10²¹ spin/g or more. With regard to the capacity of a secondary battery as many spins/g as possible is desirable.

As outlined in Scheme 1 the underlying mechanism of energy storage is the reversible oxidation/reduction of the nitroxide radical. That means during charging and discharging always two species are present, namely the nitroxide radical and its oxidized or reduced form, depending on whether it is the active material of the positive or negative electrode.

A secondary battery according to this invention has a configuration, for example, as described in EP-A-1 128 453, where a negative electrode layer and a positive electrode layer are piled via a separator containing an electrolyte. The active material used in the negative electrode layer or the positive electrode layer is generally a compound of formula Ia to Ic.

In another configuration of a laminated secondary battery a positive electrode collector, a positive electrode layer, a separator containing an electrolyte, a negative electrode layer and a negative electrode collector are piled in sequence. The secondary battery may be a multi-layer laminate as well, a combination of collectors with layers on both sides and a rolled laminate.

The negative electrode collector and the positive electrode collector may be a metal foil or metal plate made of, for example, from nickel, aluminum, copper, gold, silver, an aluminum alloy and stainless steel; a mesh electrode; and a carbon electrode. The collector may be active as a catalyst or an active material may be chemically bound to a collector. A separator made of a porous film or a nonwoven fabric may be used for preventing the above positive electrode from being in contact with the negative electrode.

An electrolyte contained in the separator transfers charged carriers between the electrodes, i.e., the negative electrode and the positive electrode, and generally exhibits an electrolyte-ion conductivity of 10⁻⁵ to 10⁻¹ S/cm at room temperature. An electrolyte used in this invention may be an electrolyte solution prepared by, for example, dissolving an electrolyte salt in a solvent. Examples of such a solvent include organic solvents such as ethylene carbonate, propylene carbonate, dimethyl carbonate, diethyl carbonate, methyl ethyl carbonate, γ-butyrolactone, tetrahydrofurane, dioxolane, sulfolane, dimethylformamide, dimethylacetamide and N-methyl-2-pyrrolidone. In this invention, these solvents may be used alone or in combination of two or more. Examples of an electrolyte salt include LiPF₆, LiClO₄, LiBF₄, LiCF₃SO₃, LiN(CF₃SO₂)₂, LiN(C₂F₆SO₂)₂, LiC(CF₃SO₂)₃ and LiC(C₂F₅SO₂)₃.

An electrolyte may be solid. Examples of a polymer used in the solid electrolyte include vinylidene fluoride polymers such as polyvinylidene fluoride, a copolymer of vinylidene fluoride and hexafluoropropylene, a copolymer of vinylidene fluoride and ethylene, a copolymer of vinylidene fluoride and monofluoroethylene, a copolymer of vinylidene fluoride and trifluoroethylene, a copolymer of vinylidene fluoride and tetrafluoroethylene and a terpolymer of vinylidene fluoride, hexafluoropropylene and tetrafluoroethylene; acrylonitrile polymers such a copolymer of acrylonitrile and methyl methacrylate, a copolymer of acrylonitrile and methyl acrylate, a copolymer of acrylonitrile and ethyl methacrylate, a copolymer of acrylonitrile and ethyl acrylate, a copolymer of acrylonitrile and methacrylic acid, a copolymer of acrylonitrile and acrylic acid and a copolymer of acrylonitrile and vinyl acetate; polyethylene oxide; a copolymer of ethylene oxide and propylene oxide; and polymers of these acrylates or methacrylates. The polymer may contain an electrolyte solution to form a gel or the polymer may be used alone.

A secondary battery in this invention may have a conventional configuration, where, for example, an electrode laminate or rolled laminate is sealed in, for example, a metal case, a resin case or a laminate film made of a metal foil such as aluminum foil and a synthetic resin film. It may take a shape of, but not limited to, cylindrical, prismatic, coin or sheet.

A secondary battery according to this invention may be prepared by a conventional process. For example, from slurry of an active material in a solvent applied on an electrode laminate. The product is piled with a counter electrode via a separator. Alternatively, the laminate is rolled and placed in a case, which is then filled with an electrolyte solution. A secondary battery may be prepared using the radical compound itself or using a compound which can be converted into the radical compound by a redox reaction, as already described above.

Another aspect of the present invention is a method for providing a secondary battery, which method comprises incorporating an active material as defined herein in at least one of the positive or negative electrodes.

Another aspect of the present invention is the use of a compound of formula Ia to Ic as defined herein as an active material in at least one of the positive or negative electrodes of a secondary battery.

Another aspect of the present invention is compound of formula Ia to Ic as defined herein, with the proviso

that G is >N⁺═OAn⁻ or
the compounds of formula Ia to Ic correspond to compounds of formula Ia1 to Ic7 (e.g. Ia1, Ia2, Ia5-Ia7, Ib1, Ib2, Ib5-Ib7, in particular Ia1, Ia2, Ia5-Ia7, Ib6, especially Ia6, Ib6) as defined above.

Preferred is a compound, which corresponds to

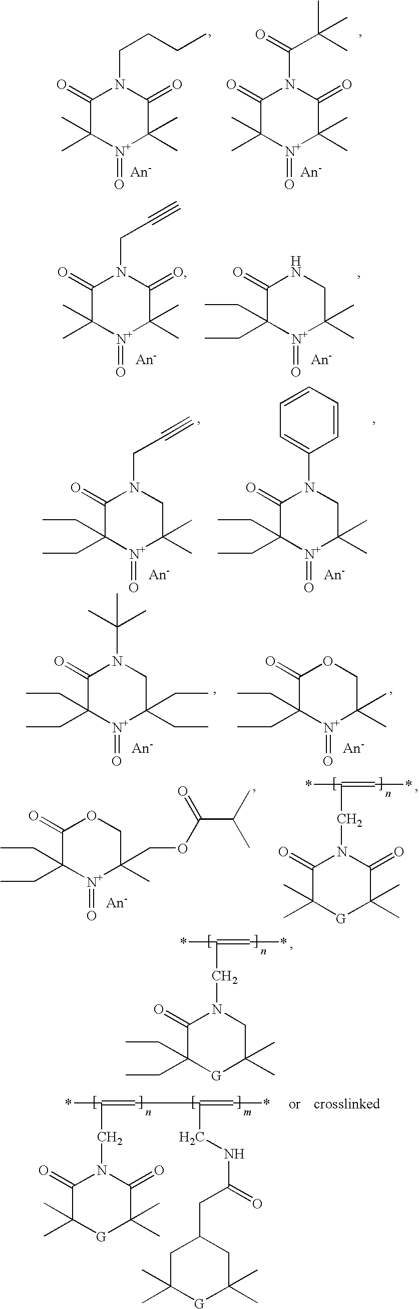

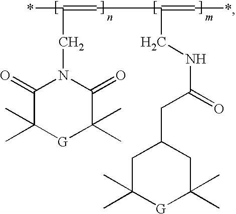

wherein m is as defined for n.

Another aspect of the present invention is a blend of a compound as defined in the preceding paragraphs and:

a further active material selected from the group consisting of an organic radical (e.g. a nitroxyl radical) different from those described herein, LiFePO₄, Li₂FeSiO₄, Li_{w}MnO₂, MnO₂, Li₄Ti₅O₁₂, LiMnPO₄, LiCoO₂, LiNiO₂, LiNi₁₋ₓCo_{y}Met_{z}O₂, LiMn_{0.5}Ni_{0.5}O₂, LiMn_{0.3}Co_{0.3}Ni_{0.3}O₂, LiFeO₂, LiMet_{0.5}Mn_{1.5}O₄, vanadium oxide, Li₁₊ₓMn_{2-z}Met_{y}O₄₋ₘXₙ, FeS₂, LiCoPO₄, Li₂FeS₂, Li₂FeSiO₄, LiMn₂O₄, LiNiPO₄, LiV₃O₄, LiV₆O₁₃, LiVOPO₄, LiVOPO₄F, Li₃V₂(PO₄)₃, MoS₃, sulfur, TiS₂, TiS₃ and combinations thereof, whereby 0<m<0.5, 0<n<0.5, 0.3≦w≦0.4, 0<x<0.3, 0<z<0.5, 0<y<0.5, Met is Al, Mg, Ti, B, Ga, Si, Ni, or Co, and X is S or F; preferably a further active material selected from the group consisting an organic radical (e.g. a nitroxyl radical) different from those described herein, LiFePO₄, MnO₂, Li₄Ti₅O₁₂, LiMnPO₄, LiCoO₂, LiMn_{0.5}Ni_{0.5}O₂, LiMn_{0.3}Co_{0.3}Ni_{0.3}O₂, vanadium oxide, FeS₂, LiMn₂O₄, LiV₃O₄, LiV₆O₁₃, sulfur, TiS₂, TiS₃ and combinations thereof,
more preferably a further active material selected from the group consisting an organic radical (e.g. a nitroxyl radical) different from those described herein, LiFePO₄, LiMnPO₄, LiCoO₂, LiMn_{0.3}Co_{0.3}Ni_{O0.3}O₂, LiMn₂O₄, and combinations thereof, for example LiFePO₄, LiMnPO₄, LiCoO₂, LiMn_{0.3}Co_{0.3}Ni_{O0.3}O₂, LiMn₂O₄, and combinations thereof,
for instance a further active material selected from the group consisting an organic radical (e.g. a nitroxyl radical) different from those described herein.

Examples of organic radicals different from those described herein are as outlined in EP-A-1128453. More particularly, the organic radical can be as represented in EP-A-1128453 as chemical formula (A1)-(A11), especially as chemical formula (A2) and (A7)-(A10), in particular as chemical formula (A7)-(A10). Further examples of organic radicals different from those described herein are as outlined in WO-A-2006/131451. More particularly, the organic radical can be a nitroxide containing polymer as outlined on page 3, line 21 to page 4, line 2. Further examples of organic radicals different from those described herein are as outlined in T. Katsumata, et al., Macromolecular Rapid Communications 2006, 27, 1206-1211. More particularly, the organic radical can be a nitroxide containing polymer denoted as poly(1)-poly(3) and as poly(4) and poly(5) as outlined on page 1207.

For instance, the weight ratio of a compound of formula Ia to Ic as defined herein to a further active material is 1:9 to 100:1, preferably 1:9 to 10:1, more preferably 1:5 to 5:1, most preferably 1:5 to 2:1.

The preferences for use, method, electrode, compound and blend are as above outlined for the secondary battery.

All % and ratio are wt.-% or weight ratio unless otherwise stated.

The following examples illustrate the invention.

### PREPARATION EXAMPLES

The compounds exemplifying the present invention are compiled in the following Table 1:

TABLE 1
Compounds

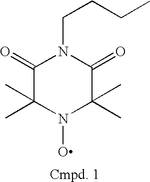

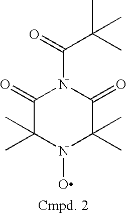

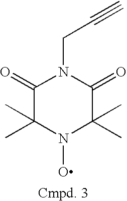

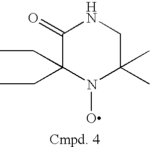

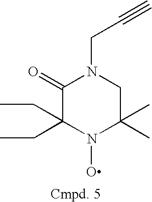

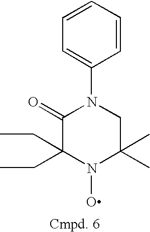

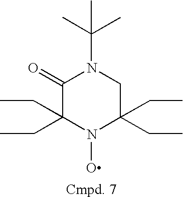

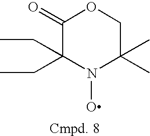

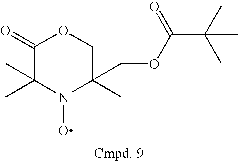

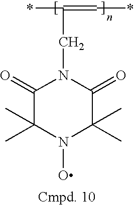

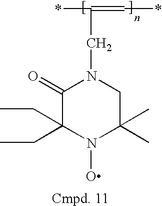

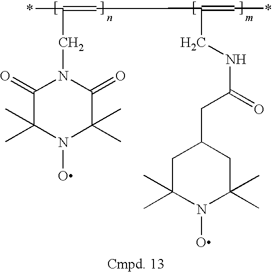

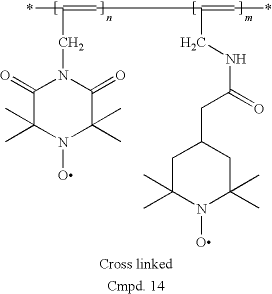

### Example 1

### (Cmpd 1): 1-Butyl-3,3,5,5-tetramethyl-piperazine-2,6-dione

Prepared in analogy to: Ramey, Chester E.; Luzzi, John J. U.S. Pat. No. 3,936,456 (1976). Red crystals, mp.=52-54° C.

### Example 2

### (Cmpd 2): 1-(2,2-Dimethyl-propionyl)-3,3,5,5-tetramethyl-piperazine-2,6-dione-4-N-oxyl

### A) 1-(2,2-Dimethyl-propionyl)-3,3,5,5-tetramethyl-piperazine-2,6-dione

3,3,5,5-tetramethyl-piperazine-2,6-dione (1.7 g, 0.01 mol, prepared according to Bulletin of the Chemical Society of Japan (1972), 45(6), 1855), triethylamine (1.6 ml, 0.011 mol) and 4-dimethylaminopyridine (55 mg) is dissolved in methylene chloride (20 ml). Then, pivaloyl chloride (1.33 g, 0.011 mol) is added during 3 minutes and the mixture is stirred for 20 h at room temperature. Methylene chloride (50 ml) and water (50 ml) is then added, the organic layer is separated and chromatographed on silica gel with dichloromethane-ethyl acetate (4:1) to afford 2.42 g of the title compound as a colorless solid, mp. 100-102° C. MS: for C₁₆H₂₃N₂O₃S (323.4) found M⁺=323.

### B) Oxidation

To a stirred mixture of 1-(2,2-dimethyl-propionyl)-3,3,5,5-tetramethyl-piperazine-2,6-dione (1.75 g, 6.88 mmol), NaHCO₃ (1.8 g, 21.4 mmol), methylene chloride (20 ml) and water (3 ml) is slowly added peracetic acid (2.1 g, 11 mmol, 40% in acetic acid) and the mixture is stirred for 17 h at room temperature. Additional 0.33 g of peracetic acid are added and the stirring is continued for 2 h. The organic layer is then separated, washed with 2 M Na₂CO₃ (2×10 ml) and evaporated. The residue is chromatographed on silica gel with dichloromethane and crystallized from hexane to afford 0.78 g of the title compound as red crystals, mp. 115-117° C. MS: for C₁₃H₂₁N₂O₄ (269.3) found M⁺=269.

### Example 3

### (Cmpd 3): 1-Propargyl-3,3,5,5-tetramethyl-piperazine-2,6-dione-4-N-oxyl

### A) 1-Propargyl-3,3,5,5-tetramethyl-piperazine-2,6-dione

To a suspension of 3,3,5,5-tetramethyl-piperazine-2,6-dione (34.04 g, 0.2 mol) (prepared according to Bulletin of the Chemical Society of Japan (1972), 45(6), 1855) in 110 ml dimethyl formamide is slowly added sodium hydride (9.6 g, 0.22 mol, 55% in mineral oil). The mixture is stirred at 40° C. until the hydrogen evolution ceases and is then cooled to 3° C. Propargyl bromide (26.2 g, 0.22 mol) is then added during 25 minutes while keeping the temperature below 10° C. The reaction is then left stirring at room temperature for 19 h. It is thereafter diluted with water (1 L) and extracted with t-butyl-methyl ether (3×150 ml). The combined extracts are dried over MgSO₄ and evaporated. The residue is crystallized from hexane to afford 27.6 g of the title compound as white crystals, mp. 77-80° C.

¹H-NMR (CDCl₃, 300 MHz), ppm: 4.53 (d, J=2.4 Hz, CH₂), 2.14 (t, J=2.4 Hz, CH), 1.44 (s, 4×CH₃).

### B) Oxidation

To a stirred mixture of 1-propargyl-3,3,5,5-tetramethyl-piperazine-2,6-dione (23.8 g, 114 mmol), NaHCO₃ (42.85 g, 510 mmol), methylene chloride (220 ml) and water (60 ml) is slowly added peracetic acid (51.7 g, 272 mmol, 40% in acetic acid) and the mixture is stirred for 4.5 h at room temperature. Additional 12.9 g of peracetic acid and 11 g NaHCO₃ are added and the stirring is continued for 2 h. The organic layer is then separated, washed with 2 M Na₂CO₃ (25 ml), water, dried over MgSO₄ and evaporated. The residue is chromatographed on silica gel with dichloromethane and crystallized from hexane to afford 22.1 g of the title compound as red crystals, mp. 92-94° C. MS: for C₁₁H₁₅N₂O₃ (223.3) found M⁺=223

### Example 4

### (Cmpd 4): 3,3-Diethyl-5,5-dimethyl-piperazin-2-one-4-N-oxyl

### A) 3,3-Diethyl-5,5-dimethyl-piperazin-2-one

1-t-Butyl-3,3-diethyl-5,5-dimethyl-piperazin-2-one (315.7 g, 1.3 mol, prepared as described in Nesvadba, Peter; Kramer, Andreas; Zink, Marie-odile. Ger. Offen. (2000), DE-A-19949352) is slowly added to hydrochloric acid (316 ml, 37%) and the mixture is refluxed for 24 h and then poured into a cold solution of NaOH (151 g, 3.775 mol) in 500 ml water. The organic layer (t-butylchloride) is discarded and the aqueous layer is extracted with t-butyl-methyl ether (5×100 ml). The combined extracts are dried over MgSO₄ and evaporated to afford crude title compound (256 g) as a yellow liquid.

### B) Oxidation

To a solution of 3,3-diethyl-5,5-dimethyl-piperazin-2-one (9.21 g, 0.05 mol) in ethyl acetate (25 ml) is slowly added peracetic acid (15.8 g, 0.083 mol, 40% in acetic acid) and the mixture is stirred for 8 h at room temperature. Water (100 ml) is then added and the mixture is extracted with t-butyl-methyl ether (6×35 ml). The extracts are washed with 5% NaOH (100 ml), dried over MgSO₄ and evaporated. The residue is recrystallized from toluene-hexane to afford 6.56 g of the title compound as yellow crystals, mp. 126-129° C. For C₁₀H₁₉N₂O₂ (199.27) calculated C, 60.27%; H, 9.61%; N, 14.05%. found C, 60.37%; H, 9.67%; N, 13.93%.

### Example 5

### (Cmpd 5): 1-Propargyl-3,3-diethyl-5,5-dimethyl-piperazin-2-one-4-N-oxyl

To a solution of 3,3-diethyl-5,5-dimethyl-piperazin-2-one-4-N-oxyl (5.98 g, 30 mmol) in dimethyl formamide (25 ml) is added sodium hydride (1.44 g, 33 mmol, 55% in mineral oil). The mixture is stirred at 40° C. until the hydrogen evolution ceases and is then cooled to 1° C. Propargyl bromide (4.92 g, 33 mol, 80% solution in toluene) is then added during 18 minutes while keeping the temperature below 8° C. The reaction is then left stirring at room temperature for 19 h. It is thereafter diluted with water (250). The solid is filtered off and recrystallized from dichloromethane-hexane to afford 4.79 g of the title compound as orange crystals, mp. 77-80° C. MS: for C₁₃H₂₁N₂O₂ (237.3) found MH⁺=238.

### Example 6

### (Cmpd 6): 1-Phenyl-3,3-diethyl-5,5-dimethyl-piperazin-2-one-4-N-oxyl

Prepared as described in Nesvadba, P., Kramer, A., Zink, M.-O.: U.S. Pat. No. 6,479,608 B1, (2002).

### Example 7

### (Cmpd 7): 1-t-Butyl-3,3,5,5-tetraethyl-piperazin-2-one-4-N-oxyl

Prepared as described in: Nesvadba, P., Kramer, A., Zink, M.-O.: U.S. Pat. No. 6,479,608 B1, (2002).

### Example 8

### (Cmpd 8): 3,3-diethyl-5,5-dimethyl-morpholin-2-one-4-N-oxyl

Prepared as described in: Nesvadba, P., Kramer, A., Zink, M.-O.: U.S. Pat. No. 6,479,608 B1, (2002).

### Example 9

### (Cmpd 9): 2,2,5-trimethyl-5-pivaloyloxymethyl-morpholin-2-one-4-N-oxyl

Prepared as described in: Nesvadba, P., Kramer, A., Zink, M.-O.: U.S. Pat. No. 6,479,608 B1, (2002).

### Example 10

### (Cmpd 10): Poly(1-propargyl-3,3,5,5-tetramethyl-piperazine-2,6-dione-4-N-oxyl

### A) Polymerization with 1 mol % of Rh(norbornadiene)BPh₄ catalyst

Solution of 1-propargyl-3,3,5,5-tetramethyl-piperazine-2,6-dione-4-N-oxyl (cmpd. 3) (4.465 g, 20 mmol) in dry dimethyl formamide (20 ml) is deoxygenated by argon purging. Thereafter, Rh(norbornadiene)BPh₄ catalyst (0.103 g, 0.2 mmol, prepared according Inorg. Chem. 1970, 9, 2339) is added at once to the vigorously stirred solution. Immediately, a slight exothermy of ca. 20° C. is observed and the reaction mixture starts to solidify. Another 30 ml of dimethyl formamide are added and the mixture is stirred for 5 h at room temperature. The semi-solid mixture is transferred into 200 ml of methanol, stirred for 1 h and filtered. The solid residue is redispersed in 200 ml methanol, stirred 12 h at room temperature and filtered. This is repeated once again, the solid polymer is then dried at 60° C./100 mbar for 16 h to afford 4.23 of the title polymer as an yellow powder.

### B) Polymerization with 0.1 mol % of Rh(norbornadiene)BPh₄ catalyst

Solution of 1-propargyl-3,3,5,5-tetramethyl-piperazine-2,6-dione-4-N-oxyl (cmpd. 3) (2.233 g, 10 mmol) in dry dimethyl formamide (25 ml) is deoxygenated by argon purging. Thereafter, Rh(norbornadiene)BPh₄ catalyst (0.0051 g, 0.01 mmol, prepared according Inorg. Chem. 1970, 9, 2339) is added at once to the vigorously stirred solution. The solution becomes turbid after ca 3 minutes. It is then stirred for 8 h at room temperature. The thick suspension is then transferred into 200 ml of methanol, stirred for 1 h and filtered. The solid residue is redispersed in 200 ml methanol, stirred 1 h at room temperature, filtered and dried at 60° C./100 mbar for 16 h to afford 1.87 of the title polymer as an yellow powder.

### C) Crosslinking polymerization with 2 mol % N,N′-dipropargyloxalamide and 0.2 mol % of Rh(norbornadiene)BPh₄ catalyst

Solution of 1-propargyl-3,3,5,5-tetramethyl-piperazine-2,6-dione-4-N-oxyl (cmpd. 3) (6.700 g, 30 mmol) and N,N′-dipropargyloxalamide (0.0985 g, 0.6 mmol) in dry dimethyl formamide (48 ml) is deoxygenated by argon purging. Thereafter, the solution of Rh(norbornadiene)BPh₄ catalyst (0.031 g, 0.06 mmol, prepared according Inorg. Chem. 1970, 9, 2339) in 2 ml dimethyl formamide is added at once to the vigorously stirred solution. The mixture becomes turbid after ca 1 h. It is then stirred for 48 h at room temperature. The thick suspension is then diluted with 50 ml of methanol, stirred for 20 h and filtered. The solid residue is redispersed in 100 ml methanol, stirred for 21 h at room temperature, filtered and dried at 60° C./100 mbar for 72 h to afford 5.892 of the title polymer as an yellow powder.

### Example 11

### (Cmpd 11): Poly(1-propargyl-3,3-diethyl-5,5-dimethyl-piperazin-2-one-4-N-oxyl)

Polymerization with 0.5 mol % of Rh(norbornadiene)BPh₄ catalyst

Solution of 1-propargyl-3,3-diethyl-5,5-dimethyl-piperazin-2-one-4-N-oxyl (cmpd. 5) (2.373 g, 10 mmol) in dry dimethyl formamide (25 ml) is deoxygenated by argon purging. Thereafter, Rh(norbornadiene)BPh₄ catalyst (0.051 g, 0.05 mmol, prepared according Inorg. Chem. 1970, 9, 2339) is added at once to the vigorously stirred solution. The mixture is then stirred for 14 h at 50° C. The thick suspension is then diluted with 300 ml of methanol, stirred for 30 minutes, filtered and dried. The solid is redisolved in 25 ml dichloromethane and precipitated by pouring into 500 ml of methanol. The precipitated polymer is filtered and dried at 50° C./100 mbar for 60 h to afford 1.72 of the title polymer as an yellow powder.

### Example 12

### (Cmpd 12) (starting compound for examples 13 and 14): N-Prop-2-ynyl-2-(2,2,6,6-tetramethyl-piperidin-4-yl)-acetamide-N-oxyl

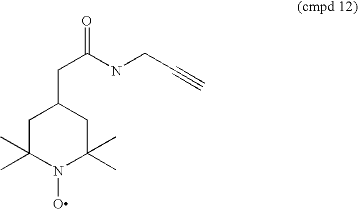

To a solution of propargylamine (0.56 g, 0.01 mol), dicyclohexylcarbodiimide (2.06 g, 0.01 mol) and 4-dimethylaminopyridine (30 mg) in 25 ml dichloromethane is added (2,2,6,6-tetramethyl-piperidin-4-yl)-acetic acid-N-oxyl (2.14 g, 0.01 mol, prepared as described in Izvestiya Akademii Nauk SSSR, Seriya Khimicheskaya 1983, (8), 1833-9). The mixture is stirred 2 h at room temperature, the precipitated dicyclohexyl urea is then filtered off, the filtrate is evaporated and the residue is recrystallized from acetonitrile to afford 1.88 g of the title compound as red crystals, m.p. 142-144° C. MS: for C₁₄H₂₃N₂O₂ (251.35) found M⁺=251.

### Example 13

### (Cmpd 13): Random copolymer of Cmpd. 3 with Cmpd. 12

Solution of 1-propargyl-3,3,5,5-tetramethyl-piperazine-2,6-dione-4-N-oxyl (cmpd. 3) (1.005 g, 4.5 mmol) and N-prop-2-ynyl-2-(2,2,6,6-tetramethyl-piperidin-4-yl)-acetamide-N-oxyl (cmpd. 12) (0.126 g, 0.5 mmol) in dry dimethyl formamide (10 ml) is deoxygenated by argon purging. Thereafter, Rh(norbornadiene)BPh₄ catalyst (0.0257 g, 0.05 mmol, prepared according Inorg. Chem. 1970, 9, 2339) is added at once to the vigorously stirred solution which is then stirred 74 h at room temperature. The mixture is then diluted with water (100 ml), the precipitate is filtered off and redispersed in methanol (50 ml). The suspension is stirred 1 h, the solid is then filtered and dried to afford 1.06 g of the title copolymer. GPC (THF, Polystyrene calibration): Mₙ=28926, M_{w}=62692.

### Example 14

### (Compound 14); Crosslinked random copolymer of Compound 3 with Compound 12

Solution of 1-propargyl-3,3,5,5-tetramethyl-piperazine-2,6-dione-4-N-oxyl (cmpd. 3) (0.893 g, 4 mmol), N-prop-2-ynyl-2-(2,2,6,6-tetramethyl-piperidin-4-yl)-acetamide-N-oxyl (cmpd. 12) (0.251 g, 1 mmol) and N,N′-dipropargyloxalamide (0.041 g, 0.25 mmol) in dry dimethyl formamide (10 ml) is deoxygenated by argon purging. Thereafter, Rh(norbornadiene)BPh₄ catalyst (0.0257 g, 0.05 mmol, prepared according Inorg. Chem. 1970, 9, 2339) is added at once to the vigorously stirred solution which is then stirred 70 h at room temperature. The mixture is then diluted with water (100 ml), the precipitate is filtered off and redispersed in methanol (50 ml). The suspension is stirred 1 h, the solid is then filtered and dried to afford 0.7 g of the title copolymer, insoluble in THF.

### B) Application Examples

### Example 101-109

### Cyclic Voltammetry

Cyclic voltammetry (CV) is performed using a three-electrode glass cell with working electrode, counter electrode and reference electrode and a computer-controlled potentiostat, applying a linear potential sweep (see e.g. B. Schoellhorn et al., New Journal of Chemistry, 2006, 30, 430-434; CAN144:441363). Multiple CV-scans per compound used are recorded and the mean value for the peak potential is taken.

### CV—Experimental Conditions

Potentiostat: VersaStat II (EG&G Instruments), 0.1M Bu₄NBF₄, 2.7E-3M nitroxide, MeCN, Pt disk d=5 mm (Working electrode), Pt wire (Counter electrode), Ag/AgCl/NaCl sat'd (Reference electrode; +0.194V vs. NHE), sweep 0-2.0V, 0.1V/s, 25° C.

The formal redox potential E₀ of the nitroxide-oxoammonium couple is calculated as the average of the anodic and cathodic peaks.

The cyclic voltammograms of selected compounds are in the Table 2

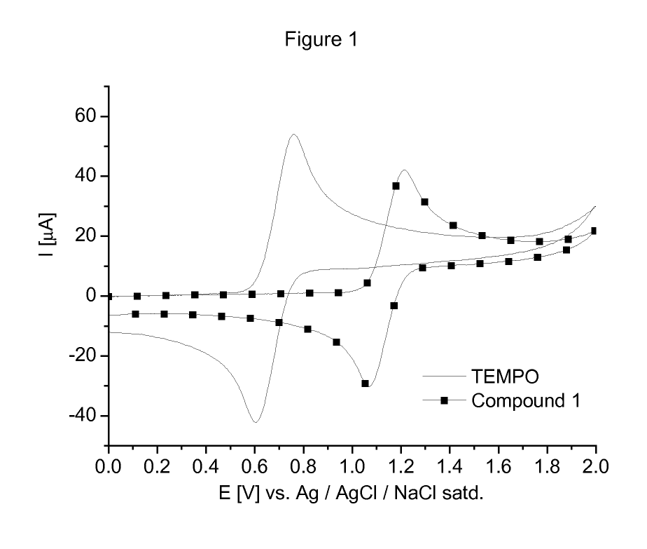

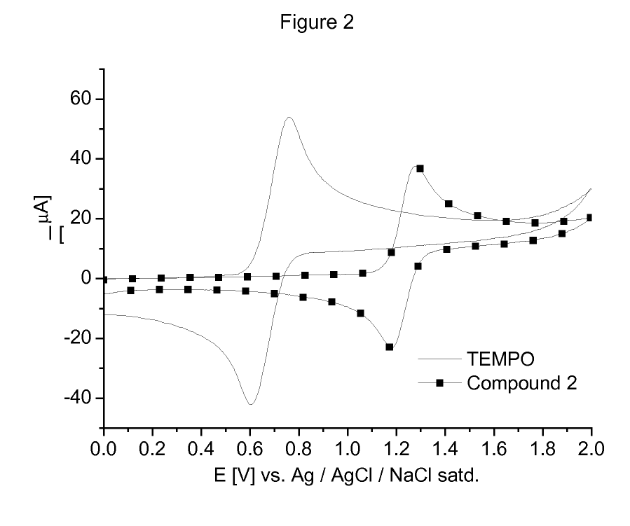

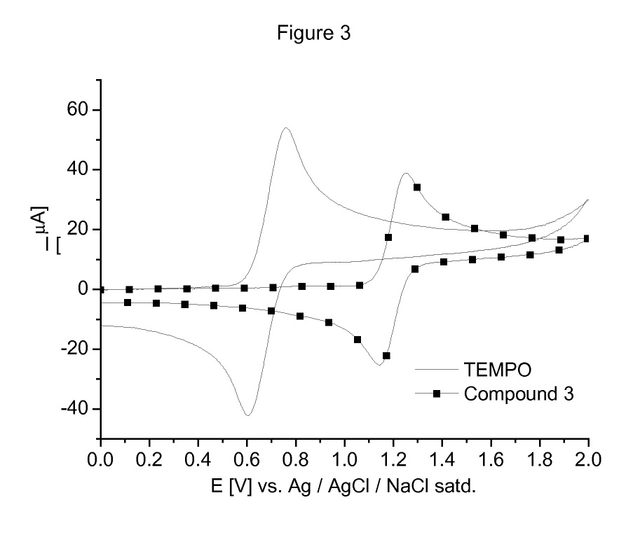

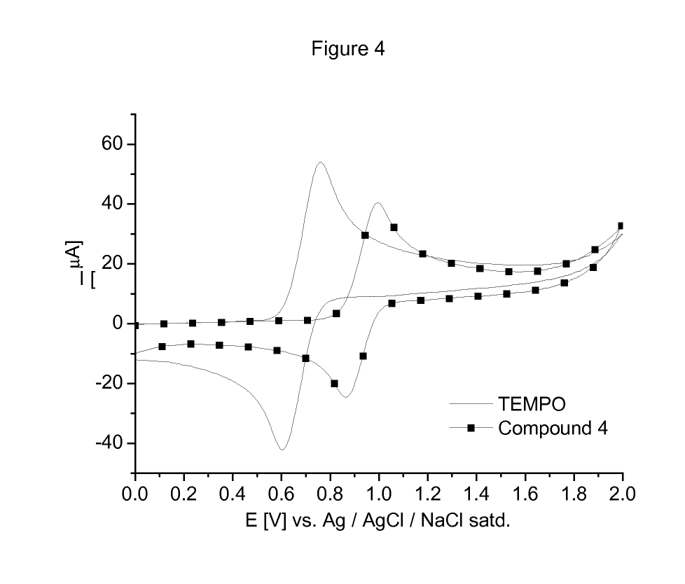

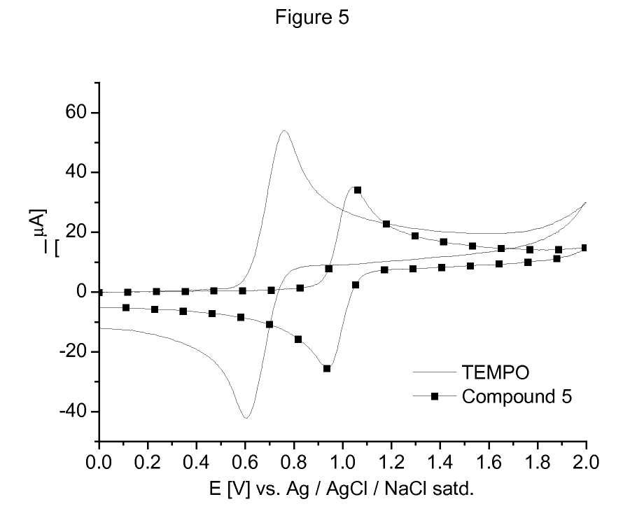

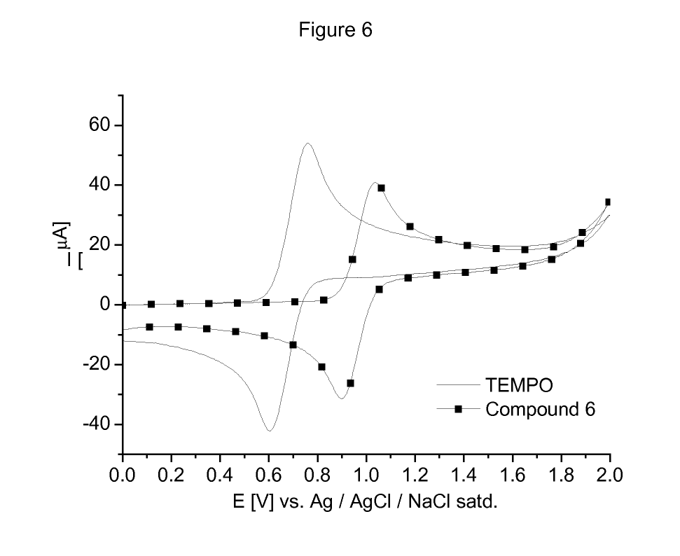

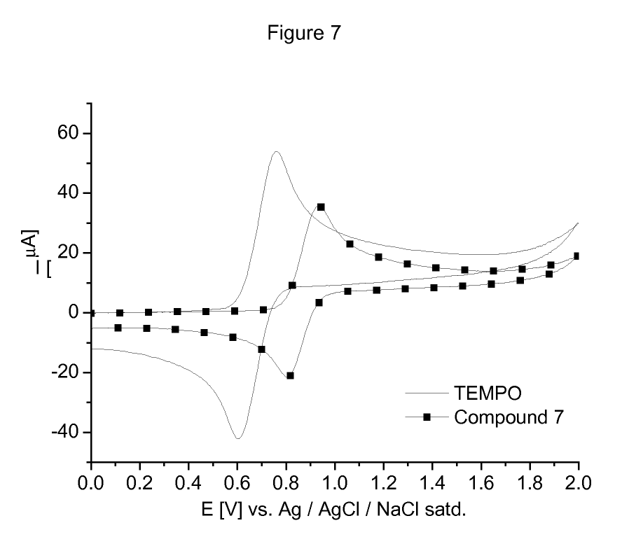

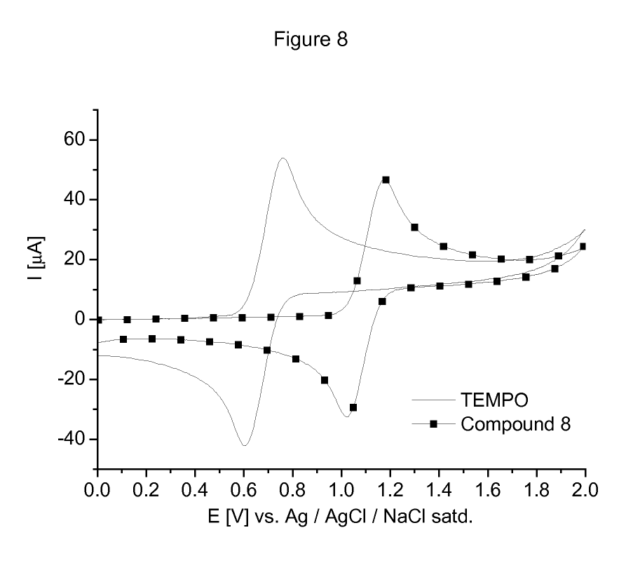

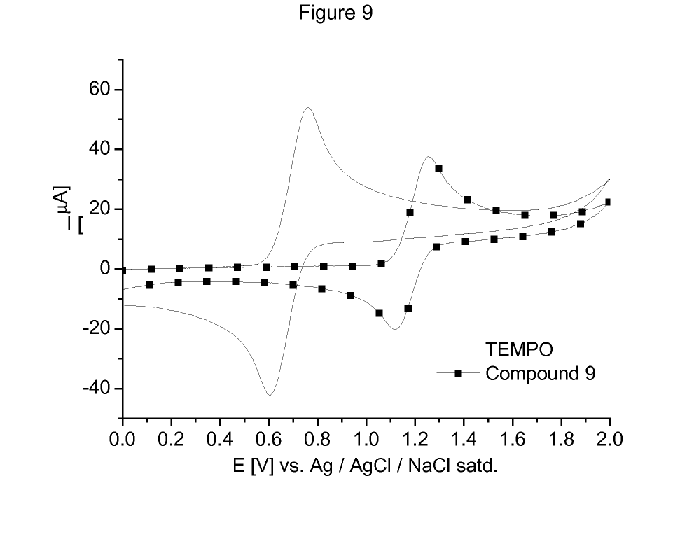

TABLE 2
Cyclic voltammograms of some examples and formal
redox potentials E₀ [V]. E₀ (TEMPO) = 0.695 V

| | |
|---|---|
| Example 101: | Cyclic voltammogram depicted in FIG. 1 |
| Cmpd 1 | |
| E₀ = 1.142 | |
| Example 102: | Cyclic voltammogram depicted in FIG. 2 |
| Cmpd 2 | |
| E₀ = 1.231 | |
| Example 103: | Cyclic voltammogram depicted in FIG. 3 |
| Cmpd 3 | |
| E₀ = 1.199 | |
| Example 104: | Cyclic voltammogram depicted in FIG. 4 |
| Reversible CV of Cmpd 4 | |
| E₀ = 0.931 | |
| Example 105: | Cyclic voltammogram depicted in FIG. 5 |
| Reversible CV of Cmpd 5 | |
| E₀ = 0.992 | |
| Example 106: | Cyclic voltammogram depicted in FIG. 6 |
| Reversible CV of Cmpd 6 | |
| E₀ = 0.969 | |
| Example 107: | Cyclic voltammogram depicted in FIG. 7 |
| Reversible CV of Cmpd. 7 | |
| E₀ = 0.871 | |
| Example 108: | Cyclic voltammogram depicted in FIG. 8 |
| Cmpd. 8 | |
| E₀ = 1.099 | |
| Example 109: | Cyclic voltammogram depicted in FIG. 9 |
| Cmpd. 9 | |
| E₀ = 1.188 | |

### Example 110

### Compound 10 in a Battery

Four parts of cmpd. 10 (prepared with 0.2 mol % of Rh(norbornadiene)BPh₄ catalyst) are thoroughly mixed with 5 parts of vapor grown carbon fibers and 1 part of poly(tetrafluoroethylene) binder. The mixture is formed by roll press into a thin electrode from which a 12 mm diameter cathode is punched out. A coin cell consisting of Lithium metal anode, ethylene carbonate—diethyl carbonate (3/7 v/v) electrolyte containing 1M LiPF₆ and separator is then assembled. The cell is then charged with 0.65 mA charging current until the electromotoric force of the cell reaches 4.15V. It is then discharged with 0.65 mA discharging current until the electromotoric force of the cell reaches 3.7 V. The delivered charge per gram of compound 10 is 114 mAh of which corresponds to 95% of the theoretical capacity (120 mAh/g) of cmpd. 10.

1. A secondary battery, utilizing an electrode reaction of an active material in the reversible oxidation/reduction cycle in at least one of the positive or negative electrodes, which active material comprises a compound of formula Ia to Ic, 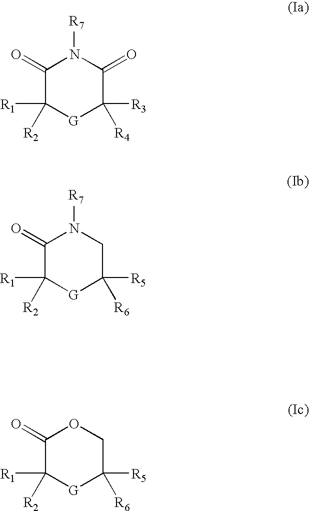
wherein G is >N—O., >N⁺═OAn⁻, >N—O⁻Li⁺ or >N—OH;
R₁, R₂, R₃, R₄, R₅ and R₆ are independently CH₃ or C₂H₅, C₅-C₆-cycloalkyl, benzyl, phenyl or
R₁ and R₂, R₃ and R₄ or R₅ and R₆ are independently together C₅- or C₆-cycloalkylidene,
or R₅ and R₆ form together with the linking carbon atom a 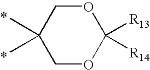
group, or
R₅ is —CH₂—X—R₁₅;
R₇ is H, OH, —CN, -halogen, C₁-C₁₈alkyl, C₆-C₁₀aryl, C₇-C₁₁aralkyl, C₂-C₁₈alkenyl, C₂-C₁₈alkynyl, C₅-C₆cycloalkyl, glycidyl, —N₃, —NH₂, —NHR₈, —NR₈R₉, —CO—OR₈, —CO—R₈, —CO—NH—R₈, —CON(R₈)(R₉), —O—CO—R₈, —SR₈, —S(═O)R₈, —S(═O)₂R₈, —S—OR₈, —S(═O)OR₈, —S(═O)₂OR₈, —SiR₈R₉R₁₀, —S(═O)₂OR₁₁ or —PO(OR₁₁)(OR₁₂),
whereby said alkyl, alkenyl, alkynyl and cycloalkyl are uninterrupted or interrupted by one or more heteroatom group selected from O, NR₈, Si(R₈)(R₉), PR₈ and S, or
whereby said alkyl, alkenyl, alkynyl and cycloalkyl are unsubstituted or substituted by one or more heteroatom group selected from F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, —NCO, —N₃, NHC(O)NHR₈, —NR₈C(O)N(R₉)(R₁₀), —NHCOOR₁₀, —N(R₈)(R₉), —NR₈COOR₁₀, —N⁺(R₈)(R₉)(R₁₀)An⁻, S⁺(R₈)(R₉)An⁻ and P⁺(R₈)(R₉)(R₁₀)An⁻, or
whereby said alkyl, alkenyl, alkynyl and cycloalkyl are interrupted by one or more heteroatom group selected from O, NR₈, Si(R₈)(R₉), PR₈ and S, and substituted by one or more heteroatom group selected from preferably by F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, —NCO, —N₃, NHC(O)NHR₈, —NR₈C(O)N(R₉)(R₁₀), —NHCOOR₁₀, —N(R₈)(R₉), —NR₈COOR₁₀, —N⁺(R₈)(R₉)(R₁₀)An⁻, S⁺(R₈)(R₉)An⁻ and P⁺(R₈)(R₉)(R₁₀)An⁻), or
R₇ is a multivalent core with one or more structural units (Ia)-(Ic) attached, or
R₇ is a 1,3,5-triazine core with 1, 2 or 3 structural units (Ia) attached;
R₈, R₉ and R₁₀ are independently H, C₁-C₁₈ alkyl, C₆-C₁₀aryl, C₇-C₁₁aralkyl, C₂-C₁₈alkenyl, C₂-C₁₈alkynyl, C₅-C₆cycloalkyl, C₄-C₁₂cycloalkenyl or C₅-C₁₂bicycloalkenyl;
R₁₁ and R₁₂ are independently H, NH₄, Li, Na, K or as defined for R₈,
R₁₃, R₁₄ are independently H or C₁-C₄ alkyl; or R₁₃ and R₁₄ form together with the linking carbon atom a C₄-C₈cycloalkylbiradical;
R₁₅ is H, C₁-C₁₈ alkyl, C₆-C₁₀aryl, C₇-C₁₁aralkyl, C₂-C₁₈alkenyl, C₂-C₁₈alkynyl, C₅-C₆cycloalkyl. glycidyl, —CO—OR₈, —CO—R₈, —CO—NH—R₈, —CON(R₈)(R₉), —S(═O)₂R₈, —S(═O)OR₈, —S(═O)₂OR₈, —SiR₈R₉R₁₀, —S(═O)₂OR₁₁ or —PO(OR₁₁)(OR₁₂),
whereby said alkyl, alkenyl, alkynyl and cycloalkyl are uninterrupted or interrupted by one or more heteroatom group selected from O, NR₈, Si(R₈)(R₉), PR₈ and S, or
whereby said alkyl, alkenyl, alkynyl and cycloalkyl are unsubstituted or substituted by one or more heteroatom group selected from F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, —NCO, —N₃, NHC(O)NHR₈, —NR₈C(O)N(R₉)(R₁₀), —NHCOOR₁₀, —N(R₈)(R₉), —NR₈COOR₁₀, —N⁺(R₈)(R₉)(R₁₀)An⁻, S⁺(R₈)(R₉)An⁻ and P⁺(R₈)(R₉)(R₁₀)An⁻, or
whereby said alkyl, alkenyl, alkynyl and cycloalkyl are interrupted by one or more heteroatom group selected from O, NR₈, Si(R₈)(R₉), PR₈ and S, and substituted by one or more heteroatom group selected from F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, —NCO, —N₃, NHC(O)NHR₈, —NR₈C(O)N(R₉)(R₁₀), —NHCOOR₁₀, —N(R₈)(R₉), —NR₈COOR₁₀, —N⁺(R₈)(R₉)(R₁₀)An⁻, S⁺(R₈)(R₉)An⁻ and P⁺(R₈)(R₉)(R₁₀)An⁻),
or R₁₅ is a multivalent core with more than one structural units (Ib)-(Ic) attached;
X is —O— or NR₁₆;
R₁₆ is as defined for R₁₅;
An⁻ is an anion of an organic or inorganic acid,
with the proviso that
R₇ does not contain a 1,3,5-triazine core for compounds of formula Ib.

2. A secondary battery according to claim 1, wherein
R₇, R₁₅ or R₁₆ as a multivalent core is a C₂-C₂₀ polyacyl of a di-, tri-, tetra-, penta- or hexa-carboxylic acid, C₂-C₂₀ alkyl, C₆-C₁₀aryl, C₅-C₉heteroaryl,
whereby said polyacyl and alkyl are uninterrupted or interrupted by one or more heteroatom group selected from O, NR₈, Si(R₈)(R₉), PR₈ and S, or
whereby said polyacyl and alkyl are unsubstituted or substituted by one or more heteroatom group selected from preferably by F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, —NCO, —N₃, NHC(O)NHR₈, —NR₈C(O)N(R₉)(R₁₀), —NHCOOR₈, —N(R₈)(R₉), —NR₈COOR₉, —N⁺(R₈)(R₉)(R₁₀) S⁺(R₈)(R₉)An⁻ and P⁺(R₈)(R₉)(R₁₀)An⁻, or
whereby said polyacyl and alkyl are interrupted by one or more heteroatom group selected from O, NR₈, Si(R₈)(R₉), PR₈ and S, and substituted by one or more heteroatom group selected from F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, —NCO, —N₃, NHC(O)NHR₈, —NR₈C(O)N(R₉)(R₁₀), —NHCOOR₈, —N(R₈)(R₉), —NR₈COOR₉, —N⁺(R₈)(R₉)(R₁₀)An⁻, S⁺(R₈)(R₉)An⁻ and P⁺(R₈)(R₉)(R₁₀)An⁻).

3. A secondary battery according to claim 2, wherein
R₁, R₂, R₃, R₄, R₅ and R₆ are independently CH₃ or C₂H₅, or
R₁ and R₂, R₃ and R₄ or R₅ and R₆ are independently together C₆-cycloalkylidene, or
R₅ is —CH₂—X—R₁₅;
R₇ is H, OH, —CN, C₁-C₆ alkyl, phenyl, benzyl, C₂-C₆alkenyl, C₂-C₆alkynyl, C₅-C₆cycloalkyl, glycidyl, —CO—OR₈, —CO—R₈, —CO—NH—R₈, —CON(R₈)(R₉), —S(═O)₂R₈, —S(═O)₂OR₈ or —PO(OR₁₁)(OR₁₂),
whereby the said alkyl, alkenyl, alkynyl and cycloalkyl are uninterrupted or interrupted by one or more O, NR₈, or S, or
whereby said alkyl, alkenyl, alkynyl or cycloalkyl are unsubstituted or substituted by one or more F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, NHC(O)NHR₈, —NR₈C(O)N(R₉)(R₁₀), —NHCOOR₁₀, —N(R₈)(R₉) or —NR₈COOR₁₀; or
whereby said alkyl, alkenyl, alkynyl and cycloalkyl are interrupted by one or more O, NR₈, or S and substituted by one or more F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, NHC(O)NHR₈, —NR₈C(O)N(R₉)(R₁₀), —NHCOOR₁₀, —N(R₈)(R₉) or —NR₈COOR₁₀;
or R₇ is a multivalent core with more than one structural units (Ib)-(Ic) attached, whereby the multivalent core is a C₂-C₈ polyacyl from di-, tri-, tetra-, penta- or hexa-carboxylic acid, C₂-C₈ alkyl or phenyl,
R₈, R₉ and R₁₀ are independently H, C₁-C₆ alkyl, phenyl, benzyl, C₂-C₄alkenyl, C₂-C₄alkynyl, C₅-C₆cycloalkyl, C₄-C₆cycloalkenyl or C₅-C₇bicycloalkenyl;
R₁₁ and R₁₂ are independently H, Li, Na, K or as defined for R₈, and
R₁₅ is H, OH, —CN, C₁-C₆ alkyl, phenyl, benzyl, C₂-C₆alkenyl, C₂-C₆alkynyl, C₅-C₆cycloalkyl, glycidyl, —CO—OR₈, —CO—R₈, —CO—NH—R₈, —CON(R₈)(R₉), —S(═O)₂R₈, —S(═O)₂OR₈ or —PO(OR₁₁)(OR₁₂),
whereby said alkyl, alkenyl, alkynyl and cycloalkyl are uninterrupted or interrupted by one or more O, NR₈, or S, or
whereby said alkyl, alkenyl, alkynyl or cycloalkyl are unsubstituted or substituted by one or more F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, NHC(O)NHR₈, —NR₈C(O)N(R₉)(R₁₀), —NHCOOR₁₀, —N(R₈)(R₉) or —NR₈COOR₁₀; or
whereby said alkyl, alkenyl, alkynyl and cycloalkyl are interrupted by one or more O, NR₈, or S and substituted by one or more F, Cl, —COOR₈, —CONHR₈, —CON(R₈)(R₉), OR₈, —OC(O)R₈, —OC(O)OR₈, —OC(O)NHR₈, —OC(O)N(R₈)(R₉), —NHC(O)R₈, —NR₈C(O)R₉, NHC(O)NHR₈, —NR₈C(O)N(R₉)(R₁₀), —NHCOOR₁₀, —N(R₈)(R₉) or —NR₈COOR₁₀.

4. A secondary battery according to claim 3, wherein
R₁, R₂, R₃, R₄, R₅ and R₆ are independently CH₃ or C₂H₅, or
R₅ is —CH₂—O—CO—C₁-C₆alkyl;
R₇ is H, C₁-C₆ alkyl, phenyl, C₂-C₃alkynyl or —CO—C₁-C₆alkyl.

5. A secondary battery according to claim 1, wherein the electrode reaction is that in the positive electrode.

6. A secondary battery according to claim 1, wherein the active material comprises a blend of at least one compound of formula Ia to Ic and a further active material selected from the group consisting of an organic radical different from Ia to Ic, LiFePO₄, Li₂FeSiO₄, Li_{w}MnO₂, MnO₂, Li₄Ti₅O₁₂, LiMnPO₄, LiCoO₂, LiNiO₂, LiNi₁₋ₓCo_{y}Met_{z}O₂, LiMn_{0.5}Ni_{0.5}O₂, LiMn_{0.3}Co_{0.3}Ni_{0.3}O₂, LiFeO₂, LiMet_{0.5}Mn_{1.5}O₄, vanadium oxide, Li₁₊ₓMn_{2-z}Met_{y}O₄₋ₘXₙ, FeS₂, LiCoPO₄, Li₂FeS₂, Li₂FeSiO₄, LiMn₂O₄, LiNiPO₄, LiV₃O₄, LiV₆O₁₃, LiVOPO₄, LiVOPO₄F, Li₃V₂(PO₄)₃, MoS₃, sulfur, TiS₂, TiS₃ and combinations thereof, whereby 0<m<0.5, 0<n<0.5, 0.3≦w≦0.4, 0<x<0.3, 0<z<0.5, 0<y<0.5, Met is Al, Mg, Ti, B, Ga, Si, Ni, or Co, and X is S or F.

7. A method for providing a secondary battery, which method comprises incorporating an active material according to claim 1 in at least one of the positive or negative electrodes.

8. (canceled)

9. A compound of formula Ia to Ic according to claim 1,
with the proviso
that G is >N⁺═OAn⁻.

10. A compound according to claim 9, which corresponds to 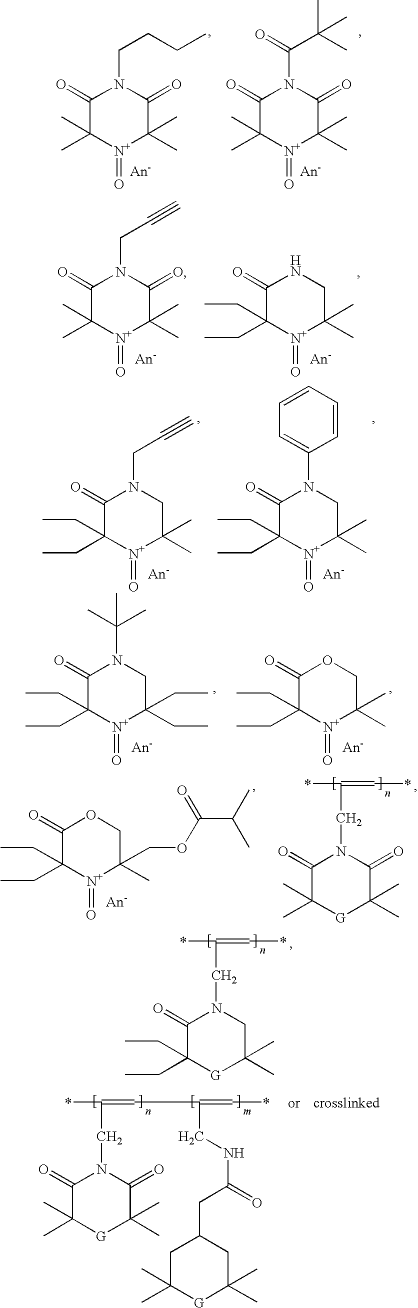 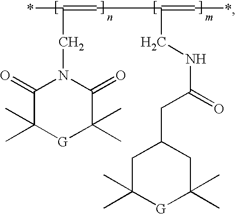
wherein m is as defined for n.

11. A blend of a compound according to claim 9 and a further active material selected from the group consisting of an organic radical different from formula Ia to Ic, LiFePO₄, Li₂FeSiO₄, Li_{w}MnO₂, MnO₂, Li₄Ti₅O₁₂, LiMnPO₄, LiCoO₂, LiNiO₂, LiNi₁₋ₓCo_{y}Met_{z}O₂, LiMn_{0.5}Ni_{0.5}O₂, LiMn_{0.3}Co_{0.3}Ni_{0.3}O₂, LiFeO₂, LiMet_{0.5}Mn_{1.5}O₄, vanadium oxide, Li₁₊ₓMn_{2-z}Met_{y}O₄₋ₘXₙ, FeS₂, LiCoPO₄, Li₂FeS₂, Li₂FeSiO₄, LiMn₂O₄, LiNiPO₄, LiV₃O₄, LiV₆O₁₃, LiVOPO₄, LiVOPO₄F, Li₃V₂(PO₄)₃, MoS₃, sulfur, TiS₂, TiS₃ and combinations thereof, whereby 0<m<0.5, 0<n<0.5, 0.3≦w≦0.4, 0<x<0.3, 0<z<0.5, 0<y<0.5, Met is Al, Mg, Ti, B, Ga, Si, Ni, or Co, and X is S or F.

12. A blend according to claim 11 comprising a compound which corresponds to 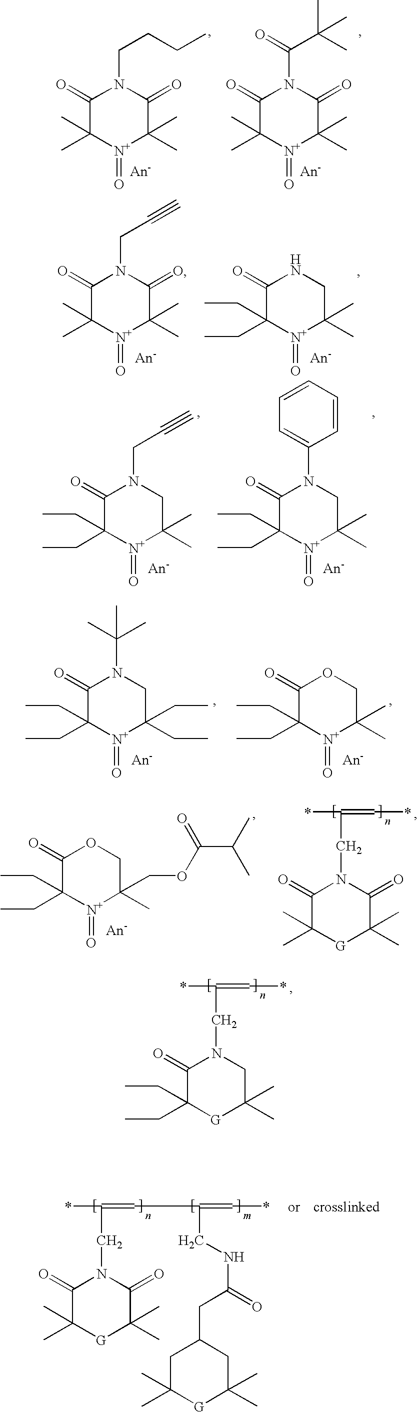 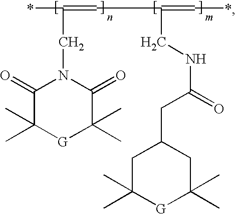
wherein m is as defined for n
and a further active material selected from the group consisting of an organic radical different from formula Ia to Ic, LiFePO₄, Li₂FeSiO₄, Li_{w}MnO₂, MnO₂, Li₄Ti₅O₁₂, LiMnPO₄, LiCoO₂, LiNiO₂, LiNi₁₋ₓCo_{y}Met_{z}O₂, LiMn_{0.5}Ni_{0.5}O₂, LiMn_{0.3}Co_{0.3}Ni_{0.3}O₂, LiFeO₂, LiMet_{0.5}Mn_{1.5}O₄, vanadium oxide, Li₁₊ₓMn_{2-z}Met_{y}O₄₋ₘXₙ, FeS₂, LiCoPO₄, Li₂FeS₂, Li₂FeSiO₄, LiMn₂O₄, LiNiPO₄, LiV₃O₄, LiV₆O₁₃, LiVOPO₄, LiVOPO₄F, Li₃V₂(PO₄)₃, MoS₃, sulfur, TiS₂, TiS₃ and combinations thereof, whereby 0<m<0.5, 0<n<0.5, 0.3≦w≦0.4, 0<x<0.3, 0<z<0.5, 0<y<0.5, Met is Al, Mg, Ti, B, Ga, Si, Ni, or Co, and X is S or F.

13. A secondary battery according to claim 1, wherein the compounds of formula Ia to Ic correspond to compounds of formula Ia1 to Ic7 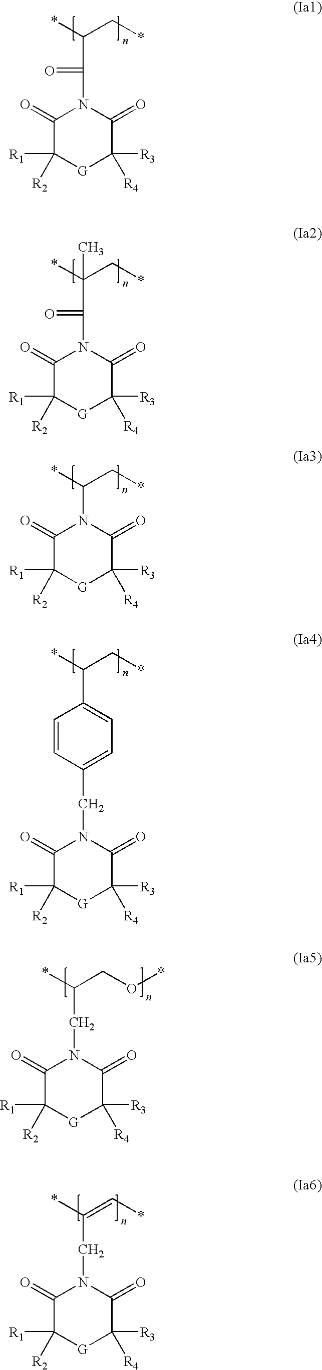 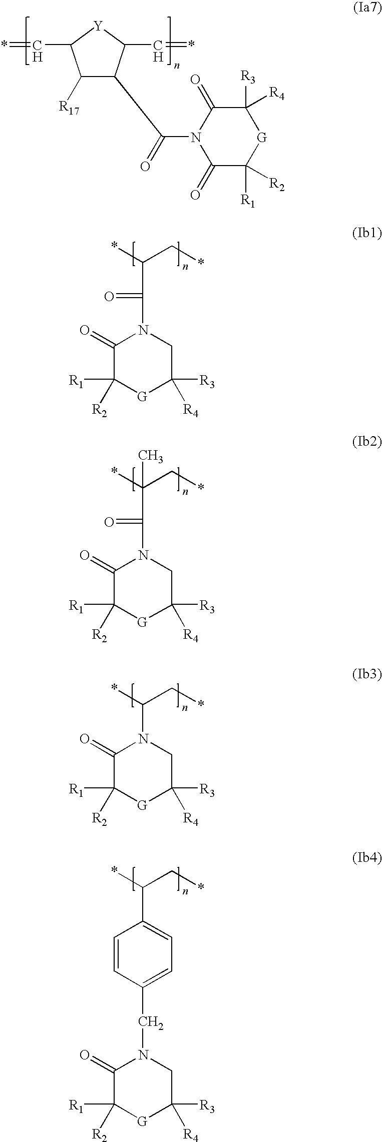 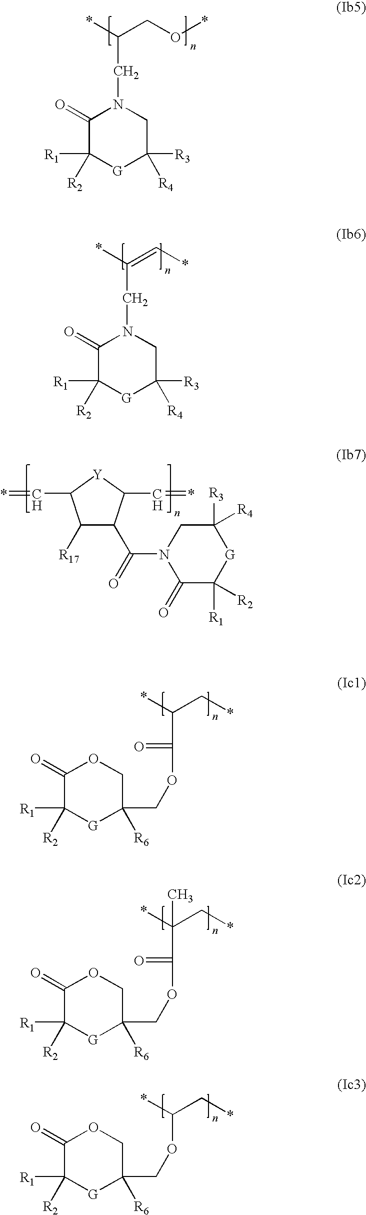 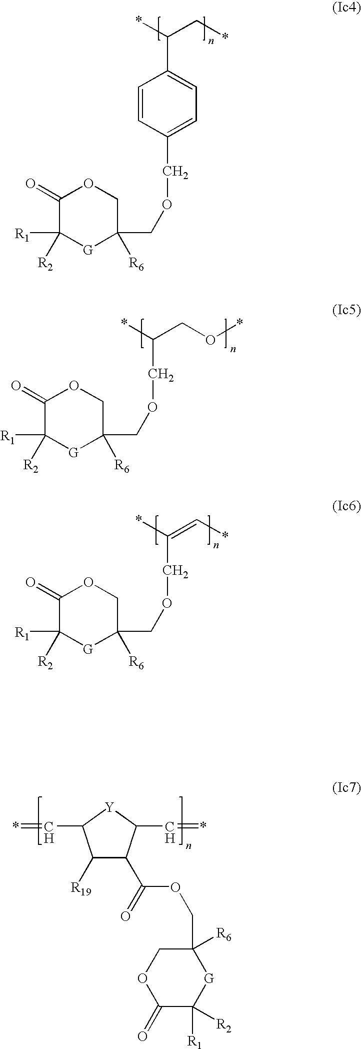
R₁₇ is H or a group 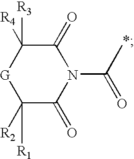
R₁₈ is H or a group 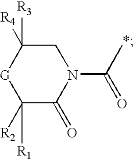
R₁₉ is H or a group 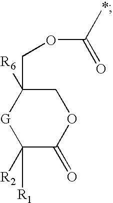
Y is —CH₂— or —O—; and
n is 2-100 000.

14. A secondary battery according to claim 13, wherein the compounds of formula Ia to Ib correspond to compounds of formula Ia1, Ia2, Ia5, Ia6, Ia7 or Ib6, wherein
R₁, R₂, R₃, and R₄ are independently CH₃ or C₂H₅,
R₁₇ is 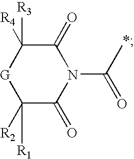
and
Y is —CH₂—.

15. A compound of formula Ia1 to Ic7 according to claim 13.

16. A secondary battery according to claim 1, wherein An⁻ is an anion derived from LiPF₆, LiClO₄, LiBF₄, LiO₃SCF₃, LiN(C₂F₅SO₂)₂, LiC(CF₃SO₂)₃, LiC(C₂F₅SO₂)₃, LiB(C₂O₄)₂, LiB(C₆H₅)₄, LiB(C₆F₅)₄, LiSbF₆, LiAsF₆, LiBr, LiBF₃C₂F₅ or LiPF₃(CF₂CF₃)₃.
